SEQUENCE LISTING (23 sequences)

SEQ ID NO: 1 - DNA, 21 bp, Homo sapiens
gccgagggagaaagggtacag

SEQ ID NO: 2 - DNA, 21 bp, Homo sapiens
tcctacatccagatgtgcagc

SEQ ID NO: 3 - DNA, 21 bp, Homo sapiens
aaggagagagagtggaggctg

SEQ ID NO: 4 - DNA, 9609 bp, Homo sapiens
tcctcctgtcctcagtggatgatcccatagaagtccagagctcggcagctgccctcccacaaaagacaggattttgaaagcagcaagagagaagagacgtatcaggtagtcacagtggctcaggcctgtaatcccagcactttgggaggcccaggtgggaggatcgcttcaccccaggaattcaagaccagcctggacaacttggaagaacccggtctctacaaaaaatacaaaattagctgggattgggtgcggtggctcatgcctataatcccagcactttgggagcctgaggtgggtggatcacctgaagtcaggagttcaagactagcctggccaacatggtgaaaccctatctctactgaaaatacaaaaagctagacgtggtggcacacacctgtaatcccagctacttaggaggctgaggcaggagaattgcttgaagcctagaggtgaaggttgtagtgagccgagattgcatcattgcacaatggaggggagccaccagcctgggcaacaagaggaaatctccgtctccaaaaaaaaaaaaaaaaaaaaaagaattaggctgggtggtgcctgtagtcccagctacttgggaggcagggggtccacttgatgtcgagactgcagtgagccatgatcctgccactgcactccggcctgggcaacagagtgagaccctgtctaaagaaaaaaaaaataaagcaacatatcctgaacaaaggatcctccataacgttcccaccagatttctaatcagaaacatggaggccagaaagcagtggaggaggacgaccctcaggcagcccgggaggatgttgtcacaggctggggcaagggccttccggctaccaactgggagctctgggaacagccctgttgcaaacaagaagccatagcccggccagagcccaggaatgtgggctgggctgggagcagcctctggacaggagtggtcccatccaggaaacctccggcatggctgggaagtggggtacttggtgccgggtctgtatgtgtgtgtgactggtgtgtgtgagagagaatgtgtgccctaagtgtcagtgtgagtctgtgtatgtgtgaatattgtctttgtgtgggtgattttctgcgtgtgtaatcgtgtccctgcaagtgtgaacaagtggacaagtgtctgggagtggacaagagatctgtgcaccatcaggtgtgtgcatagcgtctgtgcatgtcaagagtgcaaggtgaagtgaagggaccaggcccatgatgccactcatcatcaggagctctaaggccccaggtaagtgccagtgacagataagggtgctgaaggtcactctggagtgggcaggtgggggtagggaaagggcaaggccatgttctggaggaggggttgtgactacattagggtgtatgagcctagctgggaggtggatggccgggtccactgaaaccctggttatcccagaaggctttgcaggcttcaggagcttggagtggggagagggggtgacttctccgaccaggcccctccaccggcctaccctgggtaagggcctggagcaggaagcaggggcaagaacctctggagcagcccatacccgccctggcctgactctgccactggcagcacagtcaacacagcaggttcactcacagcagagggcaaaggccatcatcagctccctttataagggaagggtcacgcgctcggtgtgctgagagtgtcctgcctggtcctctgtgcctggtggggtgggggtgccaggtgtgtccagaggagcccatttggtagtgaggcaggtatggggctagaagcactggtgcccctggccgtgatagtggccatcttcctgctcctggtggacctgatgcaccggcgccaacgctgggctgcacgctacccaccaggccccctgccactgcccgggctgggcaacctgctgcatgtggacttccagaacacaccatactgcttcgaccaggtgagggaggaggtcctggagggcggcagaggtgctgaggctcccctaccagaagcaaacatggatggtgggtgaaaccacaggctggaccagaagccaggctgagaaggggaagcaggtttgggggacgtcctggagaagggcatttatacatggcatgaaggactggattttccaaaggccaaggaagagtagggcaagggcctggaggtggagctggacttggcagtgggcatgcaagcccattgggcaacatatgttatggagtacaaagtcccttctgctgacaccagaaggaaaggccttgggaatggaagatgagttagtcctgagtgccgtttaaatcacgaaatcgaggatgaagggggtgcagtgacccggttcaaaccttttgcactgtgggtcctcgggcctcactgcctcaccggcatggaccatcatctgggaatgggatgctaactggggcctctcggcaattttggtgactcttgcaaggtcatacctgggtgacgcatccaaactgagttcctccatcacagaaggtgtgacccccacccccgccccacgatcaggaggctgggtctcctccttccacctgctcactcctggtagccccgggggtcgtccaaggttcaaataggactaggacctgtagtctggggtgatcctggcttgacaagaggccctgaccctccctctgcagttgcggcgccgcttcggggacgtgttcagcctgcagctggcctggacgccggtggtcgtgctcaatgggctggcggccgtgcgcgaggcgctggtgacccacggcgaggacaccgccgaccgcccgcctgtgcccatcacccagatcctgggtttcgggccgcgttcccaaggcaagcagcggtggggacagagacagatttccgtgggacccgggtgggtgatgaccgtagtccgagctgggcagagagggcgcggggtcgtggacatgaaacaggccagcgagtggggacagcgggccaagaaaccacctgcactagggaggtgtgagcatggggacgagggcggggcttgtgacgagtgggcggggccactgccgagacctggcaggagcccaatgggtgagcgtggcgcatttcccagctggaatccggtgtcgaagtgggggcggggaccgcacctgtgctgtaagctcagtgtgggtggcgcggggcccgcggggtcttccctgagtgcaaaggcggtcagggtgggcagagacgaggtggggcaaagcctgccccagccaagggagcaaggtggatgcacaaagagtgggccctgtgaccagctggacagagccagggactgcgggagaccagggggagcatagggttggagtgggtggtggatggtggggctaatgccttcatggccacgcgcacgtgcccgtcccacccccaggggtgttcctggcgcgctatgggcccgcgtggcgcgagcagaggcgcttctccgtgtccaccttgcgcaacttgggcctgggcaagaagtcgctggagcagtgggtgaccgaggaggccgcctgcctttgtgccgccttcgccaaccactccggtgggtgatgggcagaagggcacaaagcgggaactgggaaggcgggggacggggaaggcgaccccttacccgcatctcccacccccaggacgcccctttcgccccaacggtctcttggacaaagccgtgagcaacgtgatcgcctccctcacctgcgggcgccgcttcgagtacgacgaccctcgcttcctcaggctgctggacctagctcaggagggactgaaggaggagtcgggctttctgcgcgaggtgcggagcgagagaccgaggagtctctgcagggcgagctcccgagaggtgccggggctggactggggcctcggaagagcaggatttgcatagatgggtttgggaaaggacattccaggagaccccactgtaagaagggcctggaggaggaggggacatctcagacatggtcgtgggagaggtgtgcccgggtcagggggcaccaggagaggccaaggactctgtacctcctatccacgtcagagatttcgattttaggtttctcctctgggcaaggagagagggtggaggctggcacttggggagggacttggtgaggtcagtggtaaggacaggcaggccctgggtctacctggagatggctggggcctgagacttgtccaggtgaacgcagagcacaggagggattgagaccccgttctgtctggtgtaggtgctgaatgctgtccccgtcctcctgcatatcccagcgctggctggcaaggtcctacgcttccaaaaggctttcctgacccagctggatgagctgctaactgagcacaggatgacctgggacccagcccagcccccccgagacctgactgaggccttcctggcagagatggagaaggtgagagtggctgccacggtggggggcaagggtggtgggttgagcgtcccaggaggaatgaggggaggctgggcaaaaggttggaccagtgcatcacccggcgagccgcatctgggctgacaggtgcagaattggaggtcatttgggggctaccccgttctgtcccgagtatgctctcggccctgctcaggccaaggggaaccctgagagcagcttcaatgatgagaacctgcgcatagtggtggctgacctgttctctgccgggatggtgaccacctcgaccacgctggcctggggcctcctgctcatgatcctacatccggatgtgcagcgtgagcccatctgggaaacagtgcaggggccgagggaggaagggtacaggcgggggcccatgaactttgctgggacacccggggctccaagcacaggcttgaccaggatcctgtaagcctgacctcctccaacataggaggcaagaaggagtgtcagggccggaccccctgggtgctgacccattgtggggacgcatgtctgtccaggccgtgtccaacaggagatcgacgacgtgatagggcaggtgcggcgaccagagatgggtgaccaggctcacatgccctacaccactgccgtgattcatgaggtgcagcgctttggggacatcgtccccctgggtgtgacccatatgacatcccgtgacatcgaagtacagggcttccgcatccctaaggtaggcctggcgccctcctcaccccagctcagcaccagcacctggtgatagccccagcatggctactgccaggtgggcccactctaggaaccctggccacctagtcctcaatgccaccacactgactgtccccacttgggtggggggtccagagtataggcagggctggcctgtccatccagagcccccgtctagtggggagacaaaccaggacctgccagaatgttggaggacccaacgcctgcagggagagggggcagtgtgggtgcctctgagaggtgtgactgcgccctgctgtggggtcggagagggtactgtggagcttctcgggcgcaggactagttgacagagtccagctgtgtgccaggcagtgtgtgtcccccgtgtgtttggtggcaggggtcccagcatcctagagtccagtccccactctcaccctgcatctcctgcccagggaacgacactcatcaccaacctgtcatcggtgctgaaggatgaggccgtctgggagaagcccttccgcttccaccccgaacacttcctggatgcccagggccactttgtgaagccggaggccttcctgcctttctcagcaggtgcctgtggggagcccggctccctgtccccttccgtggagtcttgcaggggtatcacccaggagccaggctcactgacgcccctcccctccccacaggccgccgtgcatgcctcggggagcccctggcccgcatggagctcttcctcttcttcacctccctgctgcagcacttcagcttctcggtgcccactggacagccccggcccagccaccatggtgtctttgctttcctggtgagcccatccccctatgagctttgtgctgtgccccgctagaatggggtacctagtccccagcctgctccctagccagaggctctaatgtacaataaagcaatgtggtagttccaactcgggtcccctgctcacgccctcgttgggatcatcctcctcagggcaaccccacccctgcctcattcctgcttaccccaccgcctggccgcatttgagacaggggtacgttgaggctgagcagatgtcagttacccttgcccataatcccatgtcccccactgacccaactctgactgcccagattggtgacaaggactacattgtcctggcatgtggggaaggggccagaatgggctgactagaggtgtcagtcagccctggatgtggtggagagggcaggactcagcctggaggcccatatttcaggcctaactcagcccaccccacatcagggacagcagtcctgccagcaccatcacaacagtcacctcccttcatatatgacaccccaaaacggaagacaaatcatggcgtcagggagctatatgccagggctacctacctcccagggctcagtcggcaggtgccagaacgttccctgggaaggccccatggaagcccaggactgagccaccaccctcagcctcgtcacctcaccacaggactggctacctctctgggccctcagggatgctgctgtacagacccctgaccagtgacgagttcgcactcagggccaggctggcgctggaggaggacacttgtttggctccaaccctaggtaccatcctcccagtagggatcaggcagggcccacaggcctgccctagggacaggagtcaaccttggacccataaggcactggggcgggcagagaaggaggaggtggcatgggcagctgagagccagagaccctgaccctagtccttgctctgccattaccccgtgtgaccccgggcccacccttccccacccttccccaccccgggcttctgtttccttctgccaacgagaaggctgcttcacctgccccgagtcctgtcttcctgctctgccttctggggctgtggcccttgctggcctggagccccaaccaagggcagggactgctgtcctccacgtctgtcctcaccgacataatgggctgggctgggcacacaggcagtgcccaagagtttctaatgagcatatgattacctgagtcctgggcagaccttcttagggaacagcctgggacagagaaccacagacactctgaggagccaccctgaggcctcttttgccagaggaccctacagcctccctggcagcagttccgccagcatttctgtaaatgccctcatgccagggtgcggcccggctgtcagcacgagagggacgttggtctgtcccctggcaccgagtcagtcagaagggtggccagggcccccttgggcccctccagagacaatccactgtggtcacacggctcggtggcaggaagtgctgttcctgcagctgtggggacagggagtgtggatgaagccaggctgggtttgtctgaagacggaggccccgaaaggtggcagcctggcctatagcagcagcaactcttggatttattggaaagattttcttcacggttctgagtcttgggggtgttagaggctcagaaccagtccagccagagctctgtcatgggcacgtagacccggtcccagggcctttgctctttgctgtcctcagaggcctctgcaaagtagaaacaggcagccttgtgagtcccctcctgggagcaaccaaccctccctctgagatgccccggggccaggtcagctgtggtgaaaggtagggatgcagccagctcagggagtggcccagagttcctgcccacccaaggaggctcccaggaaggtcaaggcacctgactcctgggctgcttccctcccctcccctccccaggtcaggaaggtgggaaagggctggggtgtctgtgaccctggcagtcactgagaagcagggtggaagcagccccctgcagcacgctgggtcagtggtcttaccagatggatacgcagcaacttccttttgaacctttttattttcctggcaggaagaagagggatccagcagtgagatcaggcaggttctgtgttgcacagacagggaaacaggctctgtccacacaaagtcggtggggccaggatgaggcccagtctgttcacacatggctgctgcctctcagctctgcacagacgtcctcgctcccctgggatggcagcttggcctgctggtcttggggttgagccagcctccagcactgcctccctgccctgctgcctcccactctgcagtgctccatggctgctcagttggacccacgctggagacgttcagtcgaagccccgggctgtccttacctcccagtctggggtacctgccacctcctgctcagcaggaatggggctaggtgcttcctcccctggggacttcacctgctctccctcctgggataagacggcagcctcctccttgggggcagcagcattcagtcctccaggtctcctgggggtcgtgacctgcaggaggaataagagggcagactgggcagaaaggccttcagagcacctcatcctcctgttctcacactggggtgtcacagtcctgggaagttcttccttttcagttgagctgtggtaaccttgtgagtttcctggagggggcctgccactacccttgggactccctgccgtgtgtctgggtctaactgagctctgaaaggagagagccccagccctgggccttccaggggaagccttacctcagaggttggcttcttcctactcttgactttgcgtctctgcagagggaggtgggaggggtgacacaaccctgacacccacactatgagtgatgagtagtcctgccccgactggcccatcctttccaggtgcagtcccccttactgtgtctgccaagggtgccagcacagccgccccactccaggggaagaggagtgccagcccttaccacctgagtgggcacagtgtagcatttattcattagcccccacactggcctgaccatctcccctgtgggctgcatgacaaggagagagaacaggctgaggtgagagctactgtcaacacctaaacctaaaaaatctataattgggctgggcagggtggctcacgcctgtaatcccagcactttgggaggccgagatgggtggatcacctgaggtcagatgttcgagaccagcctggccaacatggtgaaaccccgtctctactaaaaatacaaaaaattagctgggcgtggtggtgggtgcctgtaatcccagctactcaggaggctgaggcaggagaattgcttgaacctgggaggcagaggctgcagtgagccgagatcgcatcattgcactccagcctggtcaacaagagtgaaactgtcttaaaaaaaaaatctataattgatatctttagaaagataaaactttgcattcatgaaataagaataggagggtctaaaataaaaatgttcaaacacccaccaccactaattcttgacaaaaatatagtctgggtgccttagctcatgcctgtaatcccagcattttgggaggctaaggcaggaggattgtttgagcctaggaattc

SEQ ID NO: 5 - DNA, 20 bp, Homo sapiens
ctgtcccgagtatgctctcg

SEQ ID NO: 6 - DNA, 22 bp, Homo sapiens
ggtgtcccagcaaagttcatgg

SEQ ID NO: 7 - DNA, 25 bp, Homo sapiens
gactctgtacctcctatccacgtca

SEQ ID NO: 8 - DNA, 21 bp, Homo sapiens
gggtgtcccagcaaagttcat

SEQ ID NO: 9 - DNA, 16 bp, Homo sapiens
tcggccctgctcaggc

SEQ ID NO: 10 - DNA, 24 bp, Homo sapiens
catagtggtggctgacctgttctc

SEQ ID NO: 11 - DNA, 21 bp, Homo sapiens
ccccgttctgtcccgagtatg

SEQ ID NO: 12 - DNA, 19 bp, Homo sapiens
gctaccccgttctgtcccg

SEQ ID NO: 13 - DNA, 18 bp, Homo sapiens
gaaacagtgcaggggccg

SEQ ID NO: 14 - DNA, 19 bp, Homo sapiens
cgtgagcccatctgggaaa

SEQ ID NO: 15 - DNA, 23 bp, Homo sapiens
tgcctcctatgttggaggaggtc

SEQ ID NO: 16 - DNA, 22 bp, Homo sapiens
gccctgacactccttcttgcct

SEQ ID NO: 17 - DNA, 23 bp, Homo sapiens
tgcctcctatgttggaggaggtc

SEQ ID NO: 18 - DNA, 23 bp, Homo sapiens
ggtcaggcttacaggatcctggt

SEQ ID NO: 19 - DNA, 19 bp, Homo sapiens
ccggccctgacactccttc

SEQ ID NO: 20 - DNA, 23 bp, Homo sapiens
actgaggccttcctggcagagat

SEQ ID NO: 21 - DNA, 22 bp, Homo sapiens
atgggctcaccaggaaagcaaa

SEQ ID NO: 22 - DNA, 142 bp, Homo sapiens
  misc_feature: misc_feature CYP2D6 Exon 6
gccaaggggaaccctgagagcagcttcaatgatgagaacctgcgcatagtggtggctgacctgttctctgccgggatggtgaccacctcgaccacgctggcctggggcctcctgctcatgatcctacatccggatgtgcagc

SEQ ID NO: 23 - protein, 290 aa, Homo sapiens
MGLEALVPLAVIVAIFLLLVDLMHRRQRWAARYPPGPLPLPGLGNLLHVDFQNTPYCFDQLRRRFGDVFSLQLAWTPVVVLNGLAAVREALVTHGEDTADRPPVPITQILGFGPRSQGVFLARYGPAWREQRRFSVSTLRNLGLGKKSLEQWVTEEAACLCAAFANHSGRPFRPNGLLDKAVSNVIASLTCGRRFEYDDPRFLRLLDLAQEGLKEESGFLREVLNAVPVLLHIPALAGKVLRFQKAFLTQLDELLTEHRMTWDPAQPPRDLTEAFLAEMEKAVSNRRSTT

(19) United States
(12) Patent Application Publication
(10) Pub. No.: US 2005/0032070 A1
(43) Pub. Date: Feb. 10, 2005

(54) Polymorphisms in the human gene for CYP2D6 and their use in diagnostic and therapeutic applications

(76) Inventors: Sebastian Raimundo, Tubingen (DE); Ulrich Zanger, Korntal (DE)

Correspondence Address: FISH & NEAVE LLP, 1251 AVENUE OF THE AMERICAS, 50TH FLOOR, NEW YORK, NY, 10020-1105, US

(21) Appl. No.: 10/635,780

(22) Filed: Aug. 5, 2003

(51) Int. Cl. C12Q 1/68; C07H 21/04; C07K 14/705

(52) U.S. Cl. 435/6; 435/69.1; 435/320.1; 435/325; 530/350; 536/23.5

(57) ABSTRACT

The present invention relates to means and methods of diagnosing and treating the phenotypic spectrum as well as the overlapping clinical characteristics with several forms of inherited abnormal expression and/or function of the CYP2D6 gene. In particular, the present invention relates to polynucleotides of molecular variants of the CYP2D6 gene, which for example, are associated with abnormal drug response and disorders caused by altered activity of the CYP2D6 enzyme, and vectors comprising these polynucleotides. Furthermore, the present invention relates to host cells comprising such polynucleotides or vectors. The invention also relates to a transgenic non-human animal. Furthermore, kits comprising oligonucleotides hybridizing to the CYP2D6 gene useful for the diagnosis of an altered activity of the CYP2D6 enzyme are provided. In addition, the invention relates to methods for diagnosing a polynucleotide associated with an intermediate metabolizer phenotype of CYP2D6 drugs.

### TECHNICAL FIELD

The present invention relates to means and methods of diagnosing and treating the phenotypic spectrum as well as the overlapping clinical characteristics with several forms of inherited abnormal expression and/or function of the CYP2D6 gene. In particular, the present invention relates to polynucleotides of molecular variants of the CYP2D6 gene, which for example, are associated with abnormal drug response and disorders caused by altered activity of the CYP2D6 enzyme as well as genes and vectors comprising these polynucleotides. Furthermore, the present invention relates to host cells comprising such polynucleotides. The invention also relates to a transgenic non-human animal or solid supports comprising these polynucleotides. Furthermore, the invention encompasses compositions, such as pharmaceutical and diagnostic compositions, and a diagnostic kit. In addition, the invention relates to methods for diagnosing a polynucleotide associated with an intermediate metabolizer phenotype of CYP2D6 drugs.

Several documents are cited throughout the text of this specification. Each of the documents cited herein (including any manufacturer's specification, instructions etc.) are hereby incorporated by reference.

### BACKGROUND OF THE INVENTION

Cytochrome P450 CYP2D6 belongs to the CYP2 family of P450s and is the only functionally active isozyme of the CYP2D subfamily in humans. It is involved in the metabolism of up to 25% of all therapeutically used drugs including tricyclic antidepressants (e.g. amitriptyline, clomipramine, clopromazine, desipramine, imipramine, maprotiline, nortriptyline), selective serotonin reuptake inhibitors antidepressants (e.g. amitriptyline, clomipramine, clopromazine, desipramine, imipramine, maprotiline, nortriptyline), selective serotonin reuptake inhibitors (SSRIs) (e.g. fluoxetine, fluvoxamine, mianserine, paroxetine), antipsychotics (e.g. chlorpromazine, zotepine), neuroleptics (e.g. haloperidol, perphenazine, risperidone, thioridazine, zuclopenthizoxol), anticancer agents (e.g. tamoxifen), α-blockers (e.g. metoprolol, propanolol), antiarhythmic drugs (e.g. encainide, flecainide, mexiletine, propafenone, sparteine) and opiates (e.g. codeine, dihydrocodeine, oxycodone, tramadol).

The gene encoding its synthesis is located in the CYP2D locus at q13.1 on the long arm of chromosome 22. It is part of a gene cluster containing also two pseudogenes CYP2D7P and CYP2D8P (Kimura, Am J Hum Genet 45 (1989), 889-904).

Like other members of the human CYP2 gene family, the CYP2D genes consist of 9 exons and 8 introns. The enzyme exhibits a common genetic polymorphism (Meyer, Annu Rev Pharmacol Toxicol 37 (1997), 269-96). It was the first cytochrome P450 enzyme for which a genetic polymorphism was demonstrated which was named the debrisoquine/sparteine polymorphism based on the two substrates involved in its discovery (Eichelbaum, Eur J Clin Pharmacol 16 (1979), 183-7.; Mahgoub, Lancet 2 (1977), 584-6). Population studies have demonstrated that 5-10% of Caucasians have a severely impaired capacity to form the major metabolites 4-hydroxydebrisoquine and 2-dehydrosparteine. These subjects were defined as poor metabolizers (PMs). The remainder of the population are called extensive metabolizers (EM). The high variability in the EM enzyme activity leads to the classification of the most rapid metabolizing phenotype as ultrarapid metabolizer (UM) and the slowest of the extensive metabolizers as intermediate metabolizer (IM).

The trait ‘poor metabolism’ is inherited in an autosomal recessive fashion, i.e. PMs are carriers of two non-functional alleles. The molecular basis of this polymorphism has been extensively investigated and more than 70 functional and non-functional alleles have been described which allow to predict the PM phenotype in Caucasians with an estimated 99% reliability ((Daly, Hum Genet 95 (1995), 33741) and CYP Allele Nomenclature Web-Site: http://www.imm.ki.se/CYPalleles/cyp2d6.htm).

There are a number of possible effects of the CYP2D6 polymorphism on the metabolism and pharmacological activity of the CYP2D6 substrates. These effects depend on whether the enzyme is involved in the inactivation of pharmacologically active compounds, the bioactivation of compounds, or the formation of toxic metabolites.

Numerous case reports and clinical studies have demonstrated that for many CYP2D6 substrates, the polymorphic enzyme activity has therapeutic consequences either leading to a higher propensity to therapeutic failure at normal drug doses in UMs (e.g. prescribing of debrisoquine in UMs), to decreased drug effects in PMs (e.g. absence of analgesic effect of codeine in PMs) or even to develop adverse drug reactions and toxicity predominately in PMs. Adverse drug reactions most probably occur in the beginning of drug treatment, some even after the first dose (Kirchheiner, Acta Psychiatr Scand 104 (2001), 173-92.). PMs should therefore be very carefully dosed at the beginning of the treatment.

The IM phenotype occurs in about 10 to 15% of Caucasians who form a distinct subgroup of the population with regard to drug oxidation capacity (Griese, Pharmacogenetics 8 (1998), 15-26). Several recent studies suggest that individuals with IM phenotype may be at comparable risk as PMs (Dalen, Pharmacogenetics 9 (1999), 697-706.; Platten, Clinical Pharmacology & Therapeutics 63 (1998), 552-560), in particular under continuous therapy (Rau, Pharmacogenetics 12 (2002), 465-72.) Therefore, a genotypical differentiation between EMs and IMs for pharmacokinetic studies and individualized medicine (e.g. individualized dose adjustment) to avoid adverse drug reactions are highly desirable.

Importantly, the IM phenotype was shown to be not simply the consequence of a heterozygous condition for one null-allele and one functional allele. Only if the residual functional allele is strongly impaired compared to the wild-type allele, the consequence is intermediate (i.e. reduced) metabolic function. At least five alleles are associated with a reduction of enzymatic activity, including *9, *10, *17, *36, and *41. However, CYP2D6*9 was only found with an allelic frequency of about 1-2% across all studies, CYP2D6*10 occurs with an allelic frequency of less than 2% in the Caucasian populations, CYP2D6*17 and CYP2D6*36 have not been observed in Caucasians or are very rare alleles (Marez, Pharmacogenetics 7 (1997), 193-202; Yokota, Pharmacogenetics 3 (1993), 256-63); Broly, Pharmacogenetics 3 (1993), 123-30; Gaedigk, Pharmacogenetics 9 (1999), 669-82; Gaedigk, Clin Pharmacol Ther 72 (2002), 76-89). Therefore, these alleles can only explain a small percentage of IMs in the Caucasian population.

The *41 allele was shown to have a much higher frequency in Caucasians than the other impaired alleles, and to predict more than 50% of all IMs in such populations ((Raimundo, Pharmacogenetics 10 (2000), 577-581) and WO 00155432). This allele was almost identical to the normal functional 2D6*2 throughout all exons as well as within the gene promoter. The impaired allele *41 carried the wild-type sequence C, whereas the normally functional (EM) allele had the variant G at position −1584 G (Raimundo, Pharmacogenetics 10 (2000), 577-581). Apart from the difficulty to explain the genotype-phenotype relationship, which was assumed to involve an inhibitory NFkappa-B binding site around position −1584 bp, this constellation has the consequence that *41 could only be determined indirectly by exclusion, namely by demonstrating absence of −1584 C>G on a *2 background. In addition, this promoter polymorphism is also present in other CYP2D6 alleles and therefore the promoter polymorphism alone is not informative for phenotype prediction. Furthermore, using this marker together with polymorphisms specific for the CYP2D6*2 allele several samples have been misclassified in the past.

Accordingly, a reliable diagnosis of intermediate metabolizers (IMs) and, based on said diagnosis, diagnosing and treating a variety of forms of individual drug intolerability and inefficacy of drug therapy was hitherto not sufficiently available but are nevertheless highly desirable

Thus, the technical problem of the present invention is to provide means and methods for complying with the needs described above.

The solution to this technical problem is achieved by providing the embodiments referred to herein below and those characterized in the claims.

### SUMMARY OF THE INVENTION

The present invention relates to a polynucleotide comprising a polynucleotide selected from the group consisting of
(a) a polynucleotide having the nucleic acid sequence of SEQ ID NO: 1, 2, 3;
(b) a polynucleotide capable of hybridizing to a CYP2D6 gene, wherein said polynucleotide is having a substitution of at least one nucleotide at a position corresponding to position 4784, 4735, or 4087 of the CYP2D6 gene (SEQ ID NO 4); and
(c) a polynucleotide capable of hybridizing to a CYP2D6 gene, wherein said polynucleotide is having an A at a position corresponding to position 4784, 4735 or 4087 of the CYP2D6 gene (SEQ ID NO 4).

In a preferred embodiment, the present invention relates to polynucleotides of molecular variants of the CYP2D6 gene, wherein the nucleotide substitution is associated with an intermediate metabolizer phenotype.

In line with the foregoing, also preferably, the polynucleotide of the present invention is associated with codeine dependence, depression, hepatitis C, psychosis, schizophrenia, parkinsonism.

In a further embodiment the present invention relates to a polynucleotide which is DNA or RNA.

The invention furthermore relates to a gene comprising the polynucleotide of the invention.

In another embodiment, the present invention relates to a vector comprising the polynucleotide of the invention or the gene of the invention.

In a more preferred embodiment of the vector of the invention the polynucleotide is operatively linked to expression control sequences allowing expression in prokaryotic or eukaryotic cells or isolated fractions thereof.

The present invention furthermore relates to a host cell genetically engineered with the polynucleotide of the invention, the gene of the invention or the vector of the invention.

The present invention furthermore relates to a host cell genetically engineered with the polynucleotide of the invention, the gene of the invention or the vector of the invention.

The present invention also encompasses a transgenic non-human animal comprising at least one polynucleotide, gene or vector of the present invention.

The invention also relates to a solid support comprising one or a plurality of the polynucleotide, the gene, the vector, or the host cell of the invention in immobilized form.

In a preferred embodiment of the invention said is a membrane, a glass- or polypropylene- or silicon-chip, are membranes, oligonucleotide-conjugated beads or a bead array, which is assembled on an optical filter substrate. The term solid support is also used synonymously with the term ‘DNA chip’, ‘gene chip’, ‘GeneChip®’ (Affymetrix), other terms including ‘microarray’, ‘genome chip’ and ‘gene array’.

The invention also encompasses a composition comprising the polynucleotide, the gene, the vector or the host cell of the present invention.

In a preferred embodiment, the composition of the invention is a diagnostic composition or a pharmaceutical composition.

In a further embodiment, the present invention relates to a kit for detection of any one of the aforementioned polynucleotides, the gene, the vector, the host cell, the transgenic non-human animal or the solid support.

In a further embodiment the present invention relates to a method of diagnosing whether a subject has an extensive metabolizer (EM), intermediate metabolizer (IM) or poor metabolizer (PM) phenotype, comprising determining the absence or presence of one or more of the polynucleotides of the present invention.

Furthermore, the present invention relates to a method of diagnosing whether a subject has an EM or IM phenotype comprising determining the absence or presence of one or more of the polynucleotides of the present invention.

In an other embodiment the present invention relates to a method of diagnosing whether a subject has an IM or PM phenotype, comprising determining the absence or presence of one or more of polynucleotides of the present invention.

Furthermore, the present invention also encompasses a method of determining whether an individual is at risk for a toxic reaction to treatment with a CYP2D6 substrate which comprises determining the absence or presence of one or more of the polynucleotides of the present invention.

In another preferred embodiment the invention relates to a method of determining whether an individual is at risk for non-response to treatment with a CYP2D6 substrate which comprises determining the absence or presence of one or more of the polynucleotides of the present invention.

In a still further embodiment, the present invention relates to a method of determining whether an individual is at risk for insufficient response to treatment with a CYP2D6 substrate which comprises determining the absence or presence of one or more of the polynucleotides of the present invention.

Furthermore the present invention relates to a method of determining whether an individual is at risk for reduced metabolic activity of CYP2D6 to treatment with a CYP2D6 substrate which comprises determining the absence or presence of one or more of the of the present invention.

Another embodiment of the present invention encompasses a method of determining whether an individual is at risk for increased metabolic activity of CYP2D6 to treatment with a CYP2D6 substrate which comprises determining the absence or presence of one or more of the polynucleotides of the present invention.

In a preferred embodiment of the present invention, the above described method comprises the steps of:
(a) isolating a sample of biological material containing polynucleotides from a subject; and
(b) determining the absence or presence of one or more of the polynucleotides of the present invention.

In a further embodiment the present invention comprises a method of diagnosing whether a subject has an EM, IM or PM phenotype comprising determining the absence or presence of Exon 6 (SEQ ID No: 22) of the CYP2D6 gene (SEQ ID No: 4).

In a preferred embodiment of the invention, the method of diagnosing whether a subject has an EM or IM phenotype comprises determining the absence or presence of Exon 6 (SEQ ID No: 22) of the CYP2D6 gene (SEQ ID No: 4).

In a more preferred embodiment the invention relates to a method of diagnosing whether a subject has an IM or PM phenotype comprising determining the absence or presence of Exon 6 (SEQ ID No: 22) of the CYP2D6 gene (SEQ ID No: 4).

The present invention also encompasses a method of determining whether a subject is at risk for a toxic reaction to treatment with a CYP2D6 substrate which comprises determining the absence or presence of Exon 6 (SEQ ID No: 22) of the CYP2D6 gene (SEQ ID No: 4).

The invention also relates to a method of determining whether a subject is at risk for non-response to treatment with a CYP2D6 substrate which comprises determining the absence or presence of Exon 6 (SEQ ID No: 22) of the CYP2D6 gene (SEQ ID No: 4).

In a further embodiment the present invention comprises a method of determining whether a subject is at risk for insufficient response to treatment with a CYP2D6 substrate which comprises determining the absence or presence of Exon 6 (SEQ ID No: 22) of the CYP2D6 gene (SEQ ID No: 4).

Moreover, the present invention relates to a method of determining whether a subject is at risk for reduced metabolic activity of CYP2D6 to treatment with a CYP2D6 substrate which comprises determining the absence or presence of Exon 6 (SEQ ID No: 22) of the CYP2D6 gene (SEQ ID No: 4).

In a preferred embodiment of the present invention, the above described method comprises the steps of:
(a) isolating a sample of biological material containing polynucleotides from a subject; and
(b) determining the absence or presence of Exon 6 (SEQ ID No: 22) of the CYP2D6 gene (SEQ ID No: 4).

In a more preferred embodiment of the invention, the above described method comprises the steps of:
(a) isolating a sample of biological material containing polypeptides from a subject; and
(b) determining the absence or presence of a polypeptide having an amino acid sequence as shown in SEQ ID No: 23.

In a further preferred embodiment of the present invention, the above described method is comprising PCR based techniques, fluorescent dye and quenching agent-based PCR assay (Taqman PCR detection system), RFLP-based techniques, DNA sequencing-based techniques, hybridization techniques, single strand conformational polymorphism (SSCP), denaturating gradient gel electrophoresis (DGGE), temperature gradient gel electrophoresis (TGGE), chemical mismatch cleavage (CMC), heteroduplex analysis based system, techniques based on mass spectroscopy, invasive cleavage assay, polymorphism ratio sequencing (PRS), microarrays, a rolling circle extension assay, HPLC-based techniques, DHPLC-based techniques, oligonucleotide extension assays (OLA), extension based assays (ARMS, (Amplification Refractory Mutation System), ALEX (Amplification Refractory Mutation Linear Extension), SBCE (Single base chain extension), a molecular beacon assay, invader (Third wave technologies), a ligase chain reaction assay, 5′-nuclease assay-based techniques, hybridization capillary array electrophoresis (CAE), pyrosequencing, protein truncation assay (PTT), immunoassays and solid phase hydridization (dot blot, reverse dot blot, chips).

The present invention furthermore relates to a method of selecting a subject suffering from a CYP2D6 substrate treatable disease for a treatment with said substrate said method comprising the steps of:
(a) determining the absence or presence of one or more of the polynucleotides of the present invention.

(b) determining based on the result obtained in step (a) whether the subject can be treated with the CYP2D6 substrate wherein the absence of the polynucleotide of the present invention is indicative for a treatment.

In a more preferred embodiment the present invention encompasses a method of selecting a subject suffering from a CYP2D6 substrate treatable disease for a treatment with said substrate comprising the steps of:
(a) determining the absence or presence of absence or presence of Exon 6 (SEQ ID No: 22) of the CYP2D6 gene (SEQ ID No: 4).
(b) determining based on the result obtained in step (a) whether the subject can be treated with the CYP2D6 substrate wherein the presence of Exon 6 (SEQ ID No: 22) of the CYP2D6 gene (SEQ ID No: 4) is indicative for a treatment.

In a further embodiment the invention relates to a method of treating a subject suffering from a CYP2D6 substrate treatable disease which comprises the steps of:
(a) determining the absence or presence of one or more of the polynucleotides of the present invention.
(b) administering to a subject lacking one or more of the polynucleotides of the present invention an effective amount of the CYP2D6 substrate.

In a more preferred embodiment the invention relates to a method of treating a subject suffering from a CYP2D6 substrate treatable disease which comprises the steps of:
(a) determining the absence or presence of Exon 6 (SEQ ID No: 22) of the CYP2D6 gene (SEQ ID No: 4).
(b) administering to a subject having Exon 6 (SEQ ID No: 22) of the CYP2D6 gene (SEQ ID No: 4) an effective amount of the CYP2D6 substrate.

Furthermore, the present invention relates to a method of treating a subject suffering from a CYP2D6 substrate treatable disease which comprises the steps of:
(a) determining the absence or presence of one or more of the polynucleotides of the present invention.
(b) determining an effective amount of the CYP2D6 substrate to be used for the treatment based on the result of step (a); and
(c) in a subject having one or more of the polynucleotides of the present invention as determined in step (a), administering the amount of the CYP2D6 substrate as determined in step (b).

Preferably, the invention relates to a method of treating a CYP2D6 substrate treatable disease which comprises the steps of:
(a) determining the absence or presence of Exon 6 (SEQ ID No: 22) of the CYP2D6 gene (SEQ ID No: 4).
(b) determining an effective amount of the CYP2D6 substrate to be used for the treatment based on the result of step (a); and
(c) in a subject lacking Exon 6 (SEQ ID No: 22) of the CYP2D6 gene (SEQ ID No: 4) as determined in step (a), administering the amount of the CYP2D6 substrate as determined in step (b).

Moreover, the present invention relates to a method of identifying a diagnostic composition said method comprising the steps of:
(a) isolating a polynucleotide or the gene from a plurality of subgroups of individuals, wherein one subgroup has no prevalence for a CYP2D6 associated disease and at least one or more further subgroup(s) do have prevalence for a CYP2D6 associated disease; and
(b) identifying a single nucleotide polymorphism by comparing the nucleic acid sequence of said polynucleotide or said gene of said one subgroup having no prevalence for a CYP2D6 associated disease with said at least one or more further subgroup(s) having a prevalence for a CYP2D6 associated disease.

The invention relates to a method of diagnosing a disease related to the presence of a molecular variant of a CYP2D6 gene or susceptibility to such a disorder comprising determining the presence of a polynucleotide of the invention in a sample from a subject.

Moreover, the invention relates to a method of detection of the polynucleotide or the gene of the invention in a sample comprising the steps of:
(a) contacting the solid support described supra with the sample under conditions allowing interaction of the polynucleotide or the gene with the plurality of immobilized targets on the solid support and;
(b) determining the binding of said polynucleotide or said gene to said immobilized targets on the solid support.

The invention also relates to a method for diagnosing a disease comprising the steps of the method described supra, wherein binding of said polynucleotide or gene to said immobilized targets on said solid support is indicative for the presence or the absence of said disease or a prevalence for said disease.

In a preferred embodiment of the above described method said disease is codeine dependence, depression, hepatitis C, psychosis, schizophrenia, parkinsonism.

The novel variant forms of CYP2D6 gene according to the invention provide the potential for the development of a pharmacodynamic profile of drugs for a given patient.

Figure Legends

The following figures illustrate the invention:

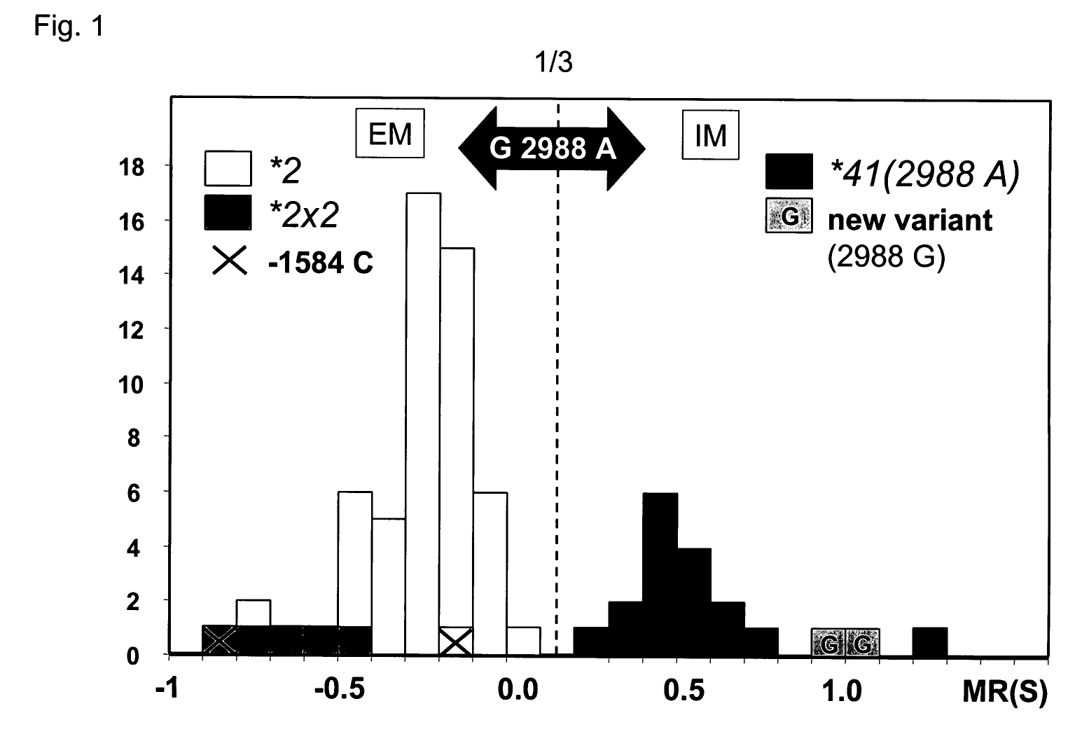

FIG. 1: Distribution of the variant 2988G>A dependent on the in vivo phenotype for sparteine oxidation in the Caucasian population

The histogram shows the distribution of the sparteine metabolic ratio (on a log scale) in the population. The white bars corresponds to individuals with variant allele *2 (2988G), grey bars to individuals with the allele *2×*2 (2988G), X to individuals with the *2 allele and −1584 C, black bars to individuals with the allele *41 (2988A), grey G bars to individuals with the new variants 2939G>A, 2291G>A (2988G). EM: extensive metabolizer (range MRs<1,2); IM: intermediate metabolizer (range: 1.2<MRs<20), y-axis: number of individuals.

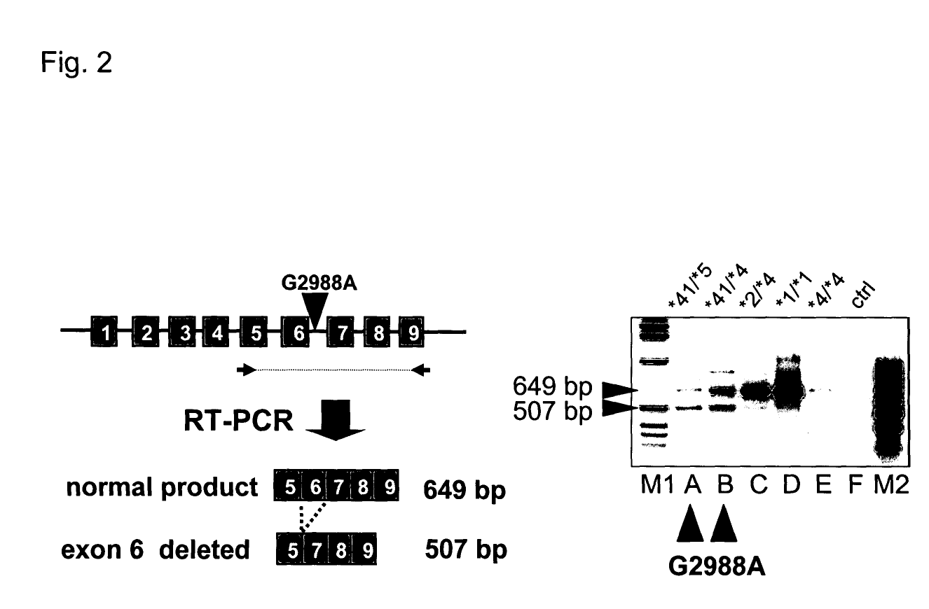

FIG. 2: Analysis of CYP2D6 splice variants in human liver

Left side: Strategy for RT-PCR of CYP2D6 for exon 5-exon 9. Right side: agarose gel electrophoresis of RP-PCR products for the normal (wild type) 649 bp product and the 507 bp splice variant product for individuals with different genotypes. A: sample 99, genotyp (*41/*5); B: sample 17, genotyp (*41/*4); C: sample 7, genotyp (*2(−1584 G)/*4); D: sample 18, genotyp (*1/*1); E: sample 112, genotyp (*4/*4), F: negative control; M1: 1 kb ladder; M2: 100 bp ladder.

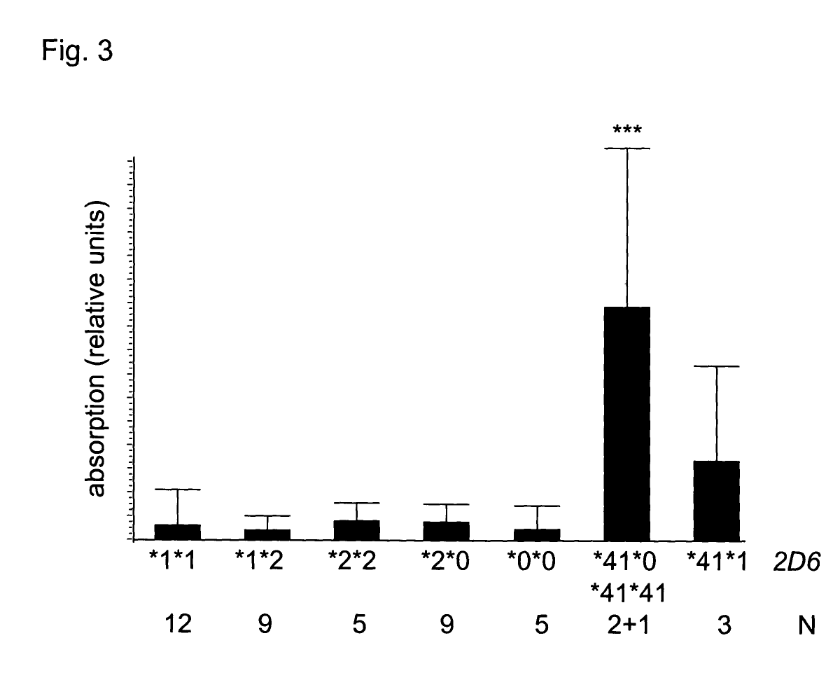

FIG. 3: Quantification of CYP2D6 splice variant by DHPLC

The DHPLC method was used for quantification of the splice variant of CYP2D6 lacking exon 6. The relative absorption of the 507 bp fragment normalized to 18S rRNA was compared between groups of different CYP2D6 genotypes: *1/*1 (n=12), *1/*2 (n=9), *2/*2 (n=5), *2/*0 (n=9), *0/*0 (n=5), *41/*0 (n=2), *41/*41 (n=1), *41/*1 (n=3).

***p<0,001 vs *1/*1, *1/*2, *21*2, *2/*0, *0/*0 (one-way Anova)

### DETAILED DESCRIPTION OF THE INVENTION

The finding of variations in the CYP2D6 gene, and diagnostic test for the discrimination of different genetic variants of the CYP2D6 gene in human individuals provides a very potent tool for improving drug therapy of diseases which are treated with drugs that are metabolized by the CYP2D6 drug metabolizing enzyme. This diagnosis of the individuals genetic constitution of the CYP2D6 status may be used for personalized medicine (e.g. individual dose regime dependent on the genetic status of the individual). It may also be used for prediction of the therapy outcome of an individual with an established drug and for avoidance of side effects/toxicity due to an altered activity of CYP2D6 mediated by different CYP2D6 alleles. Furthermore, diagnostic tests to genotype CYP2D6 will help to correlate the genotypes with the drug activity and evaluate the clinical outcome of these drugs, with may lead to adverse effects and drug interactions.

The present invention provides a way to exploit molecular biology and pharmaceutical research for drug therapy while bypassing their potential detrimental effects which are due to expression of variant expression of the CYP2D6 gene.

Accordingly, the present invention relates to a polynucleotide comprising a polynucleotide selected from the group consisting of
(a) a polynucleotide having the nucleic acid sequence of SEQ ID NO: 1, 2, 3;
(b) a polynucleotide capable of hybridizing to a CYP2D6 gene, wherein said polynucleotide is having a substitution of at least one nucleotide at a position corresponding to position 4784, 4735, or 4087 of the CYP2D6 gene (SEQ ID NO 4); and
(c) a polynucleotide capable of hybridizing to a CYP2D6 gene, wherein said polynucleotide is having an A at a position corresponding to position 4784, 4735 or 4087 of the CYP2D6 gene (SEQ ID NO 4).

In the context of the present invention the term “polynucleotides” or the term “polypeptides” refers to different variants of a polynucleotide. Said variants comprise a reference or wild type sequence of the polynucleotides or polypeptides of the invention as well as variants which differ therefrom in structure or composition. Reference or wild type sequences for the polynucleotides is SEQ ID NO 4. The differences in structure or composition usually occur by way of nucleotide substitution(s). The variant polynucleotides also comprise fragments of said polynucleotides of the invention. The polynucleotides as well as the aforementioned fragments thereof of the present invention are characterized as being associated with a reduced activity of the CYP2D6 enzyme. Such a reduced activity can be determined by techniques well known in the art, e.g., by measuring the sparteine oxidation, the dextromethorphan and debrisoquine metabolic ratio as described in Bock, Pharmacogenetics 4 (1994), 209-218; Griese, Pharmacogenetics 8 (1998), 15-26 and Sachse, Am J Hum Genet 60 (1997), 284-295. Said altered activity referred to in the present invention cause a reduced clearance of a drug metabolized by the drug metabolizing enzyme CYP2D6. Such a reduced clearance can be determined by techniques well known in the art, e.g., Malcolm (1995): Clinical Pharmacokinetics: Concepts and Applications. Lippincott, Williams & Wilkins, ISBN:0683074040 and Goodman & Gillman's, The Pharmacological Basis of Therapeutics, Mc Graw Hill Medical Publishing Division (2001) ISBN: 0-07-135469-7. Total clearance is defined as the volume of blood cleared of the drug by the various elimination processes (metabolism and excretion) per unit time.

Dependent whether a drug itself or its metabolite is the active substance this will lead to a higher plasma concentration of the drug or lower plasma concentration of the metabolite and therefore increases the risk of developing adverse drug reaction or toxicity of the drug and/or metabolite.

The term “hybridizing” as used herein refers to polynucleotides which are capable of hybridizing to the polynucleotides of the invention or parts thereof which are associated with altered expression of the variant CYP2D6 gene compared to the wild type gene. Thus, said hybridizing polynucleotides are also associated with said altered expression. Therefore, said polynucleotides may be useful as probes in Northern or Southern Blot analysis of RNA or DNA preparations, respectively, or can be used as oligonucleotide primers in PCR analysis dependent on their respective size. Also comprised by the invention are hybridizing polynucleotides which are useful for analysing DNA-Protein interactions via, e.g., electrophoretic mobility shift analysis (EMSA). Preferably, said hybridizing polynucleotides comprise at least 10, more preferably at least 15 nucleotides in length while a hybridizing polynucleotide of the present invention to be used as a probe preferably comprises at least 100, more preferably at least 200, or most preferably at least 500 nucleotides in length.

It is well known in the art how to perform hybridization experiments with nucleic acid molecules, i.e. the person skilled in the art knows what hybridization conditions she has to use in accordance with the present invention. Such hybridization conditions are referred to in standard text books such as Molecular Cloning A Laboratory Manual, Cold Spring Harbor Laboratory (1989) N.Y. Preferred in accordance with the present inventions are polynucleotides which are capable of hybridizing to the polynucleotides of the invention or parts thereof which are associated with an altered CYP2D6 expression under stringent hybridization conditions, i.e. which do not cross hybridize to unrelated polynucleotides such as polynucleotides that may not alter the expression of the CYP2D6 gene compared to the corresponding wild type gene.

Nucleic acid hybridization will be affected by such conditions as salt concentration, temperature, or organic solvents, in addition to the base composition, length of the complementary strands and the number of nucleotide base mismatches between the hybridizing nucleic acids, as will be readily appreciated by those skilled in the art. Stringent temperature conditions will generally include temperatures in excess of 30° C., typically 37° C., and preferably in excess of 45° C. Stringent salt conditions will ordinarily be less than 1000 mM, typically less than 500 mM and preferably less than 200 mM. However, the combination of parameters is much more important than the measure of any single parameter. See, e.g., Wetmur and Davidson, 1968. Probe sequences may also hybridize specifically to duplex DNA under certain conditions to form triplex or higher order DNA complexes. The preparation of such probes and suitable hybridization conditions are well known in the art. Typically hybridizing polynucleotides and probes which are used have a sequence binding to the polymorphism of interest, and the flanking sequence, in a specific manner and thus typically have a sequence which is fully or partially complementary to the sequence of the polymorphism and the flanking region. The hybridizing polynucleotides of this invention encompass a polynucleotide (i) a polynucleotide which can be specifically amplified from a sample comprising the CYP2D6 gene (SEQ ID No: 4) by using a first oligonucleotide upstream from the nucleotides 4784, 4735, or 4087 of the CYP2D6 gene (SEQ ID No: 4) and a second oligonucleotide derived from the reverse complementary nucleic acid sequence downstream of nucleotides 4784, 4735, or 4087 of the CYP2D6 gene (SEQ ID No: 4), wherein said polynucleotide is having a substitution of at least one nucleotide at a position corresponding to position 4784, 4735, or 4087 of the CYP2D6 gene (SEQ ID NO 4) or (ii) a polynucleotide which can be specifically amplified from a sample comprising the CYP2D6 gene (SEQ ID No: 4) by using a first oligonucleotide upstream from the nucleotides 4784, 4735, or 4087 of the CYP2D6 gene (SEQ ID No: 4) and a second oligonucleotide derived from the reverse complementary nucleic acid sequence downstream of nucleotides 4784, 4735, or 4087 of the CYP2D6 gene (SEQ ID No: 4), wherein said polynucleotide is having an A at a position corresponding to position 4784, 4735 or 4087 of the CYP2D6 gene (SEQ ID NO 4).

The term “specifically amplified” refers to methods of nucleic acid amplification wherein a nucleic acid fragment which is flanked by the oligonucleotide sequences is amplified and the reaction does not yield significant amounts of unrelated nucleic acid amplification products. Whether unrelated amplification products do occur can be tested, e.g., by carrying out a control amplification reaction in which merely one of the oligonucleotides in used. Preferably, the amplification methods are PCR-based methods wherein a fragment of a polynucleotide is amplified by a DNA polymerization reaction. The principles of PCR are well known in the art and are described in detail in standard text books of molecular biology. The PCR-based method encompass in particular those referred to below. In accordance with the present invention, most preferably, the PCR is carried as specified in Example 1 in order to result in specific amplification.

The term “sample” encompasses samples of biological material comprising CYP2D6 polynucleotides. Such samples may be tissue samples, blood samples, liquor samples, samples of excretion products and other body fluids. Alternatively, isolated cells, such as cultivated cells, tissue culture cells or dispersed cells from tissues may be used as samples. The samples may be treated by techniques well known in the art in order to allow amplification of the polynucleotides. The person skilled in the art can select a suitable treatment without further ado depending on the nature of the polynucleotide to be amplified. For instance, in a sample comprising genomic DNA to be used as a template for the amplification reaction, said genomic DNA may be extracted first from the tissue cells by standard methods. Alternatively, if, e.g., a sample containing RNA is used, it will be necessary to transcribe said RNA in cDNA before amplification can be carried out. Again, techniques for doing so are well known to the person skilled in the art.

The term “oligonucleotide”, in principle, refers to polynucleotide molecules consisting of 15 to 50, preferably 18 to 40, more preferably 18 to 28 nucleotides in length. In accordance with the present invention, the oligonucleotides may comprise all types of chemical modifications as long as these modifications do not interfere with proper base pairing and, thus, with the specificity of said oligonucleotides in the amplification reaction.

The term “derived” means that an oligonucleotide as specified hereinabove has a nucleic acid sequence which comprises between 15 to 50, preferably 18 to 40, more preferably 18 to 28 continuous nucleotides in length of the flanking sequences of the polynucleotide fragment to be amplified. Specifically, for the amplification reaction referred to hereinabove, the first oligonucleotide must comprise a sequence having 15 to 50, preferably 18 to 40, more preferably 18 to 28 continuous nucleotides in length of nucleotides upstream of nucleotides 4784, 4735, or 4087 of the CYP2D6 gene (SEQ ID No: 4). The second oligonucleotide must comprise a sequence having 15 to 50, preferably 18 to 40, more preferably 18 to 28 continuous nucleotides in length of nucleotides downstream of nucleotides 4784, 4735, or 4087 of the CYP2D6 gene (SEQ ID No: 4). Most preferably, the oligonucleotides derived from said sequences of the CYP2D6 gene have a nucleic acid sequence for the first and for the second oligonucleotide as indicated in Example 1, wherein the first primer corresponds to the “forward primer” referred to in the Example and the second primer to the “reverse primer”.

The term “corresponding” as used herein means that a position is not only determined by the number of the preceding nucleotides and amino acids, respectively. The position of a given nucleotide in accordance with the present invention which may be substituted may vary due to deletions or additional nucleotides elsewhere in the gene or the polypeptide. Thus, under a “corresponding position” in accordance with the present invention it is to be understood that nucleotides may differ in the indicated number but may still have similar neighboring nucleotides. Said nucleotides which may be exchanged are also comprised by the term “corresponding position”. Said nucleotides or amino acids may for instance together with their neighbors form sequences which may be involved in the regulation of gene expression, stability of the corresponding RNA or RNA editing, as well as encode functional domains or motifs of the protein of the invention.

In accordance with the present invention, three new genetic variants in the CYP2D6 gene have been identified and the mode and population distribution of a genetic variation in intron 6 of the CYP2D6 gene has been analyzed by sequence analysis of relevant regions of the human said gene from many different individuals. It is a well known fact that genomic DNA of individuals, which harbor the individual genetic makeup of all genes, including the CYP2D6 gene, can easily be purified from individual blood samples. These individual DNA samples are then used for the analysis of the sequence composition of the alleles of the CYP2D6 gene that are present in the individual which provided the blood sample. The sequence analysis was carried out by PCR amplification of relevant regions of said gene, subsequent purification of the PCR products, followed by automated DNA sequencing with established methods (e.g. ABI dyeterminator cycle sequencing, DNA analysis system from Licor and/or DHPLC analysis); see also examples.

This work represents a mutation analysis within intronic regions of the CYP2D6 gene that has not been analyzed before and the identification of unknown genetic variations in exonic regions. The aim was to identify genetic variants within unknown regulatory elements (e.g enhancer or silencer elements, splicing enhancer, splicing silencer elements), that are linked to a low expression of the CYP2D6 gene and thus lead to a reduced activity of the CYP2D6 enzyme. Such a reduced metabolic activity of the CYP2D6 enzyme leads to a severely impaired ability to metabolize CYP2D6 substrates and this phenotype is termed intermediate metabolizer (IM) phenotype. Genotyping methods so far are only indirect methods analyzing several genetic variant positions in parallel and therefore the genotyping methods encompass many independent steps that need to be combined resulting into a cost intensive genotyping procedure. In addition, it is necessary to establish genetic variants that can be used for a precise phenotype-genotype prediction without any false predictions for efficient and safe drug therapy.

In accordance with the present invention three mutations have been identified which are associated with an intermediate metabolizer phenotype of CYP2D6 (i.e. reduced activity of the CYP2D6 enzyme). Particularly, the mutation 2988G>A has been associated with a higher urinary metabolic ratio for sparteine oxidation, described as an intermediate metabolizer phenotype of CYP2D6. This allelic variant has an allelic frequency of 8,75% to 12,2% in the random Caucasian population and now allows a direct establishing of the genotype for the identification of an intermediate metabolizer phenotypes in the Caucasian population. Thus, the polynucleotides of the present invention are, preferably, molecular variants of the CYP2D6 gene, wherein the nucleotide substitution results in reduced expression and/or reduced activity of the variant CYP2D6 gene compared to the corresponding wild type. A reduced expression and/or reduced activity in accordance with the use of the present invention means that the expression of the wild type allele differs significantly from the expression of the variant allele. A significant difference can be determined by standard statistical methods, such as Student's t-test, chi²-test or the U-test according to Mann and Whitney. Moreover, the person skilled in the art can adopt these and other statistical method known in the art individually without an undue burden. Advantageously, the characterization of said mutants leads to an improved diagnostic method for the identification of individuals with an intermediate metabolizer phenotype with a reduction of false diagnostic predictions.

One important parameter that had to be considered in the attempt to determine the individual genotypes and identify novel variants of the CYP2D6 gene by direct DNA-sequencing of PCR-products from human blood genomic DNA is the fact that each human harbors (usually, with very few abnormal exceptions) two gene copies of each autosomal gene (diploidy). Because of that, great care had to be taken in the evaluation of the sequences to be able to identify unambiguously not only homozygous sequence variations but also heterozygous variations. The details of the different steps in the identification and characterization of novel polymorphisms in the CYP2D6 gene (homozygous and heterozygous) are described in the examples below.

The methods of the mutation analysis followed standard protocols and are described in detail in the examples. In general such methods are to be used in accordance with the present invention for evaluating the phenotypic spectrum as well as the overlapping clinical characteristics of plasma concentration of drugs metabolized by CYP2D6 or altered response to drugs in patients with the mutation including non response, inefficacy, adverse drug effects or toxicity encompass for example haplotype analysis, single-strand conformation polymorphism analysis (SSCA), PCR, HPLC, TaqMan technology and direct sequencing. On the basis of thorough clinical characterization of many patients the phenotypes can then be correlated to these newly identified mutations and mutation earlier described.

Over the past 20 years, genetic heterogeneity has been increasingly recognized as a significant source of variation in drug response. Many scientific communications (Meyer, Ann. Rev. Pharmacol. Toxicol. 37 (1997), 269-296 and West, J. Clin. Pharmacol. 37 (1997), 635-648) have clearly shown that some drugs work better or may even be highly toxic in some patients than in others and that these variations in patient's responses to drugs can be related to molecular basis. This “pharmacogenomic” concept spots correlations between responses to drugs and genetic profiles of patient's (Marshall, Nature Biotechnology, 15 (1997), 954-957; Marshall, Nature Biotechnology, 15 (1997), 1249-1252). In this context of population variability with regard to drug therapy, pharmacogenomics has been proposed as a tool useful in the identification and selection of patients which can respond to a particular drug without side effects. This identification/selection can be based upon molecular diagnosis of genetic polymorphisms by genotyping DNA from leukocytes in the blood of patient. For the founders of health care, such as health maintenance organizations in the US and government public health services in many European countries, this pharmacogenomics approach can represent a way of both improving health care and reducing overheads because there is a large cost to unnecessary drugs, ineffective drugs and drugs with side effects.

As is evident to the person skilled in the art, the genetic knowledge deduced from the present invention can now be used to exactly and reliably characterize the genotype of a patient. Advantageously, diseases or a prevalence for a disease which are associated with CYP2D6 dysfunction or dysregulation, such as codeine dependence, depression, hepatitis C, psychosis, schizophrenia, parkinsonism referred to herein can be predicted and preventive or therapeutical measures can be applied accordingly. Moreover in accordance with the foregoing, in cases where a given drug takes an unusual effect, a suitable individual therapy can be designed based on the knowledge of the individual genetic makeup of a subject with respect to the polynucleotides of the invention and improved therapeutics can be developed as will be further discussed below.

Finally, the polynucleotides and polypeptides referred to in accordance with the present invention are also useful as forensic markers, which improve the identification of subjects which have been murdered or killed by, for example a crime of violence or any other violence and can not be identified by the well known conventional forensic methods. The application of forensic methods based on the detection of the polymorphisms comprised by the polynucleotides of this invention in the genome of a subject are particularly well suited in cases where a (dead) body is disfigured in a severe manner such as identification by other body characteristics such as the features of the face is not possible. This is the case, for example, for corpses found in water which are usually entirely disfigured. Advantageously, methods which are based on the provision of the polynucleotides of the invention merely require a minimal amount of tissue or cells in order to be carried out. Said tissues or cells may be blood droplets, hair roots, epidermal scales, saliva droplets, sperms etc. Since only such a minimal amount of tissue or cells is required for the identification of a subject, the polymorphism comprised by the polynucleotides of this invention can also be used as forensic markers in order to proof someone guilty for a crime, such as a violation or a ravishment. Moreover, the polymorphisms comprised by the polynucleotides of this invention can be used to proof paternity. In accordance with the forensic methods referred herein the presence or absence of the polynucleotides of the invention is determined and compared with a reference sample which is unambiguously derived from the subject to be identified. The forensic methods which require detection of the presence or absence of the polynucleotides of this invention in a sample of a subject the polymorphisms comprised by the polynucleotides of this invention can be for example PCR-based techniques which are particularly well suited in cases where only minimal amount of tissue or cells is available as forensic samples. On the other hand, where enough tissue or cells is available, hybridization based techniques may be performed in order to detect the presence or absence of a polynucleotide of this invention. These techniques are well known by the person skilled in the art and can be adopted to the individual purposes referred to herein without further ado. In conclusion, thanks to the present invention forensic means which allow improved and reliable predictions as regards the aforementioned aspects are now available.

In a preferred embodiment, the present invention relates to polynucleotides of molecular variants of the CYP2D6 gene, wherein the nucleotide substitution is associated with an intermediate metabolizer phenotype.

The term “intermediate metabolizer phenotype” refers to a an individual with an intermediate function of the CYP2D6 enzyme as described in (Griese, Pharmacogenetics 8 (1998), 15-26; Raimundo, Pharmacogenetics 2000 10 (7) (2000), 577-581; Bock, Pharmacogenetics 4 (1994), 209-18). The in vivo activity of the CYP2D6 enzyme can be determined for example using different probe drugs such as sparteine, debrisoquine, dextrometorphan and metoporolol (Levy, (2000), Metabolic drug interactions, Lippincot Williams & Wilkins, ISDN 0-77817-1441-9). The metabolic activity can be obtained by determination of the metabolic ratio (MR) for the probe drug used (Griese, Pharmacogenetics 8 (1998), 15-26). The distribution of the MR among is not unimodally distributed but can be separated in at least different groups: extensive metabolizer (IM), intermediate metabolizer (IM) and poor metabolizer (PM). For sparteine (Griese, Pharmacogenetics 8 (1998), 15-26) have defined that the MR for EMs is MR<20; for IM 1,2<MR<20 and for PMs MR>20.

In line with the foregoing, also preferably, the polynucleotide of the present invention is associated with codeine dependence, depression, hepatitis C, psychosis, schizophrenia, parkinsonism.

The terms “codeine dependence, depression, hepatitis C, psychosis, schizophrenia, parkinsonism” used herein are very well known and characterized in the art. The symptoms are described in standard text books of medicine such as Harrisons's Principles of internal medicine 15^{th} edition (2001), McGraw Hill SBN 0-07-0025113490 and Stedman. The clinical practitioner can determine the aforementioned diseases if occurring in a subject based on the symptoms without further ado.

In a further embodiment the present invention relates to a polynucleotide which is DNA or RNA.

The polynucleotide of the invention may be, e.g., DNA, cDNA, genomic DNA, RNA or synthetically produced DNA or RNA or a recombinantly produced chimeric nucleic acid molecule comprising any of those polynucleotides either alone or in combination. Preferably said polynucleotide is part of a vector, particularly plasmids, cosmids, viruses and bacteriophages used conventionally in genetic engineering that comprise a polynucleotide of the invention. Such vectors may comprise further genes such as marker genes which allow for the selection of said vector in a suitable host cell and under suitable conditions.

The invention furthermore relates to a gene comprising the polynucleotide of the invention.

It is well known in the art that genes comprise structural elements which encode an amino acid sequence as well as regulatory elements which are involved in the regulation of the expression of said genes. Structural elements are represented by exons which may either encode an amino acid sequence or which may encode for RNA which is not encoding an amino acid sequence but is nevertheless involved in RNA function, e.g. by regulating the stability of the RNA or the nuclear export of the RNA.

Regulatory elements of a gene may comprise promoter elements or enhancer elements both of which could be involved in transcriptional control of gene expression. It is very well known in the art that a promoter is to be found upstream of the structural elements of a gene. Regulatory elements such as enhancer elements, however, can be found distributed over the entire locus of a gene. Said elements could be reside, e.g., in introns, regions of genomic DNA which separate the exons of a gene. Promoter or enhancer elements correspond to polynucleotide fragments which are capable of attracting or binding polypeptides involved in the regulation of the gene comprising said promoter or enhancer elements. For example, polypeptides involved in regulation of said gene comprise the so called transcription factors.

Said introns may comprise further regulatory elements which are required for proper gene expression. Introns are usually transcribed together with the exons of a gene resulting in a nascent RNA transcript which contains both, exon and intron sequences. The intron encoded RNA sequences are usually removed by a process known as RNA splicing. However, said process also requires regulatory sequences present on a RNA transcript said regulatory sequences may be encoded by the introns.

In addition, besides their function in transcriptional control and control of proper RNA processing and/or stability, regulatory elements of a gene could be also involved in the control of genetic stability of a gene locus. Said elements control, e.g., recombination events or serve to maintain a certain structure of the DNA or the arrangement of DNA in a chromosome.

Therefore, single nucleotide polymorphisms can occur in exons of a gene which encode an amino acid sequence as discussed supra as well as in regulatory regions which are involved in the above discussed process. The analysis of the nucleotide sequence of a gene locus in its entirety including, e.g., introns is in light of the above desirable. The polymorphisms comprised by the polynucleotides of the present invention can influence the expression level of CYP2D6 protein via mechanisms involving reduced transcription of the CYP2D6 gene, stabilization of the gene's RNA transcripts and alteration of the processing of the primary RNA transcripts. In particular, the polynucleotide of the invention relates to a polymorphism in intron 6 of the CYP2D6 gene that leads to altered splicing which results in CYP2D6 transcripts lacking exon 6 and therefore premature termination of the CYP2D6 protein. As a consequence the expression and metabolic activity of the wild-type CYP2D6 protein is reduced in an individual carrying such a polynucleotide in comparison to those with the wild-type polynucleotide.

Therefore, in a furthermore preferred embodiment of the gene of the invention a nucleotide substitution results in altered expression of the variant gene compared to the corresponding wild type gene.

In another embodiment, the present invention relates to a vector comprising the polynucleotide of the invention or the gene of the invention.

Said vector may be, for example, a phage, plasmid, viral or retroviral vector. Retroviral vectors may be replication competent or replication defective. In the latter case, viral propagation generally will occur only in complementing host/cells.

The polynucleotides or genes of the invention may be joined to a vector containing selectable markers for propagation in a host. Generally, a plasmid vector is introduced in a precipitate such as a calcium phosphate precipitate, or in a complex with a charged lipid or in carbon-based clusters. Should the vector be a virus, it may be packaged in vitro using an appropriate packaging cell line prior to application to host cells.

In a more preferred embodiment of the vector of the invention the polynucleotide is operatively linked to expression control sequences allowing expression in prokaryotic or eukaryotic cells or isolated fractions thereof.

Expression of said polynucleotide comprises transcription of the polynucleotide, preferably into a translatable mRNA. Regulatory elements ensuring expression in eukaryotic cells, preferably mammalian cells, are well known to those skilled in the art. They usually comprise regulatory sequences ensuring initiation of transcription and optionally poly-A signals ensuring termination of transcription and stabilization of the transcript. Additional regulatory elements may include transcriptional as well as translational enhancers. Possible regulatory elements permitting expression in prokaryotic host cells comprise, e.g., the lac, trp or tac promoter in E. coli, and examples for regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in bacculo, mammalian and other animal cells. Beside elements which are responsible for the initiation of transcription such regulatory elements may also comprise transcription termination signals, such as the SV40-poly-A site or the tk-poly-A site, downstream of the polynucleotide. In this context, suitable expression vectors are known in the art such as Okayama-Berg cDNA expression vector pcDV1 (Pharmacia), pCDM8, pRc/CMV, pcDNA1, pcDNA3 (In-vitrogene), pSPORT1 (GIBCO BRL). Preferably, said vector is an expression vector and/or a gene transfer or targeting vector. Expression vectors derived from viruses such as retroviruses, vaccinia virus, adeno-associated virus, herpes viruses, or bovine papilloma virus, may be used for delivery of the polynucleotides or vector of the invention into targeted cell population. Methods which are well known to those skilled in the art can be used to construct recombinant viral vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994). Alternatively, the polynucleotides and vectors of the invention can be reconstituted into liposomes for delivery to target cells.

The term “isolated fractions thereof” refers to fractions of eukaryotic or prokaryotic cells or tissues which are capable of transcribing or transcribing and translating RNA from the vector of the invention. Said fractions comprise proteins which are required for transcription of RNA or transcription of RNA and translation of said RNA into a polypeptide. Said isolated fractions may be, e.g., nuclear and cytoplasmic fractions of eukaryotic cells such as of reticulocytes.

The present invention furthermore relates to a host cell genetically engineered with the polynucleotide of the invention, the gene of the invention or the vector of the invention.

The present invention furthermore relates to a host cell genetically engineered with the polynucleotide of the invention, the gene of the invention or the vector of the invention.

The present invention also encompasses a method for the production of a transgenic non-human animal comprising introduction of a polynucleotide or vector of the invention into a germ cell, an embryonic cell, stem cell or an egg or a cell derived therefrom. The non-human animal can be used in accordance with the method of the invention described below and may be a non-transgenic healthy animal, or may have a disease or disorder, preferably a disease caused by at least one mutation in the gene of the invention. Such transgenic animals are well suited for, e.g., pharmacological studies of drugs in connection with variant forms of the above described variant polypeptides since these polypeptides or at least their functional domains are conserved between species in higher eukaryotes, particularly in mammals. Production of transgenic embryos and screening of those can be performed, e.g., as described by A. L. Joyner Ed., Gene Targeting, A Practical Approach (1993), Oxford University Press. The DNA of the embryos can be analyzed using, e.g., Southern blots with an appropriate probe or based on PCR techniques.

A transgenic non-human animal in accordance with the invention may be a transgenic mouse, rat, hamster, dog, monkey, rabbit, pig, frog, nematode such as Caenorhabditis elegans, fruit fly such as Drosophila melanogaster or fish such as torpedo fish or zebrafish comprising a polynucleotide or vector of the invention or obtained by the method described above, preferably wherein said polynucleotide or vector is stably integrated into the genome of said non-human animal, preferably such that the presence of said polynucleotide or vector leads to the expression of the variant polypeptide of the invention. It may comprise one or several copies of the same or different polynucleotides or genes of the invention. This animal has numerous utilities, including as a research model for cardiovascular research and therefore, presents a novel and valuable animal in the development of therapies, treatment, etc. for diseases caused by cardiovascular diseases. Accordingly, in this instance, the mammal is preferably a laboratory animal such as a mouse or rat.

Thus, in a preferred embodiment the transgenic non-human animal of the invention is a mouse, a rat or a zebrafish.

Numerous reports revealed that said animals are particularly well suited as model organisms for the investigation of the drug metabolism and its deficiencies or cancer. Advantageously, transgenic animals can be easily created using said model organisms, due to the availability of various suitable techniques well known in the art.

The invention also relates to a transgenic non-human animal comprising at least one polynucleotide of the invention, the gene of the invention or the vector of the invention as described supra.

The invention also relates to a solid support comprising one or a plurality of the polynucleotide, the gene, the vector, or the host cell of the invention in immobilized form.

The term “solid support” as used herein refers to a flexible or non-flexible support that is suitable for carrying said immobilized targets. Said solid support may be homogenous or inhomogeneous. For example, said solid support may consist of different materials having the same or different properties with respect to flexibility and immobilization, for instance, or said solid support may consist of one material exhibiting a plurality of properties also comprising flexibility and immobilization properties. Such supports are well known in the art and comprise, inter alia, commercially available column materials, polystyrene beads, latex beads, magnetic beads, colloid metal particles, glass and/or silicon chips and surfaces, nitrocellulose strips, membranes, sheets, duracytes, wells and walls of reaction trays, plastic tubes etc. Examples of well-known carriers include glass, polystyrene, polyvinyl chloride, polypropylene, polyethylene, polycarbonate, dextran, nylon, amyloses, natural and modified celluloses, polyacrylamides, agaroses, and magnetite. Preferably, said solid support may comprise glass-, polypropylene- or silicon-chips, membranes oligonucleotide-conjugated beads or bead arrays.

The term “immobilized” means that the molecular species of interest is fixed to a solid support, preferably covalently linked thereto. This covalent linkage can be achieved by different means depending on the molecular nature of the molecular species. Moreover, the molecular species may be also fixed on the solid support by electrostatic forces, photolithography, hydrophobic or hydrophilic interactions or Van-der-Waals forces. The above described physico-chemical interactions typically occur in interactions between molecules. For example, biotinylated polypeptides may be fixed on a avidin-coated solid support due to interactions of the above described types. Further, polypeptides such as antibodies, may be fixed on an antibody coated solid support. Moreover, the immobilization is dependent on the chemical properties of the solid support. For example, the nucleic acid molecules can be immobilized on a membrane by standard techniques such as UV-crosslinking, photolithography or heat. Moreover, it is very well known in the art how these solid supports can be applied in various methods including those specifically referred to in accordance with the present invention (see, e.g., Syvänen, Nature Reviews 2 (2001), 930-942)

In a preferred embodiment of the invention said solid support is a membrane, a glass- or polypropylene- or silicon-chip, are membranes oligonucleotide-conjugated beads or a bead array, which is assembled on an optical filter substrate.

The invention also encompasses a composition comprising the polynucleotide, the gene, the vector or the host cell of the present invention.

In a preferred embodiment, the composition of the invention is a diagnostic composition or a pharmaceutical composition.

The term “diagnostic composition” comprises at least one of the aforementioned compounds of the invention in soluble form or liquid phase but it is also envisaged that said compounds are immobilized on a solid support as specified above. The solid supports of the present invention may be used in combination with the diagnostic composition as defined herein or the compounds of the present invention may be used as diagnostic compositions in immobilized form on said solid supports. The compounds to be used as diagnostic compositions may be labeled with one or more second compounds. Said second compounds used as labels may be either directly or indirectly detectable. Suitable labels which are directly detectable encompass compounds which have, e.g., fluorescent properties. Suitable labels which are indirectly detectable comprise one or more chemical groups which can be converted from a status in which they cannot be detected directly into a status in which they can be detected directly or the label itself may be detected by a further compound, such as an antibody, which itself is detectably labeled and thus allows detection of the compound used as an indirect label. Appropriate labels and methods for labeling are well known in the art and furthermore mentioned herein below. A variety of techniques are available for labeling biomolecules, are well known to the person skilled in the art and are considered to be within the scope of the present invention. Such techniques are, e.g., described in Tijssen, “Practice and theory of enzyme immuno assays”, Burden, R H and von Knippenburg (Eds), Volume 15 (1985), “Basic methods in molecular biology”; Davis L G, Dibmer M D; Battey Elsevier (1990), Mayer et al., (Eds) “Immunochemical methods in cell and molecular biology” Academic Press, London (1987), or in the series “Methods in Enzymology”, Academic Press, Inc. There are many different labels and methods of labeling known to those of ordinary skill in the art. Examples of the types of labels which can be used in the present invention include enzymes, radioisotopes, colloidal metals, fluorescent compounds, chemiluminescent compounds, and bioluminescent compounds. Commonly used labels comprise, inter alia, fluorochromes (like fluorescein, rhodamine, Texas Red, etc.), enzymes (like horse radish peroxidase, β-galactosidase, alkaline phosphatase), radioactive isotopes (like ³²P or ¹²⁵I), biotin, digoxygenin, colloidal metals, chemi- or bioluminescent compounds (like dioxetanes, luminol or acridiniums). Labeling procedures, like covalent coupling of enzymes or biotinyl groups, iodinations, phosphorylations, biotinylations, etc. are well known in the art. Detection methods comprise, but are not limited to, nucleic acid hybridization techniques, such as Southern Blots, Northern Blots, South-Western Blots, autoradiography based techniques in general, fluorescence microscopy, direct and indirect enzymatic reactions, etc. Commonly used detection assays comprise radioisotopic or non-radioisotopic methods. These comprise, inter alia, Westernblotting, overlay-assays, RIA (Radioimmuno Assay) and IRMA (Immune Radioimmunometric Assay), EIA (Enzyme Immuno Assay), ELISA (Enzyme Linked Immuno Sorbent Assay), FIA (Fluorescent Immuno Assay), and CLIA (Chemioluminescent Immune Assay). Moreover, detection methods may include PCR-based techniques such as those referred to herein below.

It is particularly preferred that the diagnostic composition of the invention is employed for determining whether a subject has an IM, PM or EM phenotype and/or is at risk, has a prevalence for or will develop a disease or disorder as referred to in accordance with the present invention.

The term “pharmaceutical composition” comprises the substances of the present invention and optionally one or more pharmaceutically acceptable carrier. The substances of the present invention may be formulated as pharmaceutically acceptable salts. Substances comprising e.g. the antibody may conveniently be administered by any of the routes conventionally used for drug administration, for instance, orally, topically, parenterally or by inhalation. Acceptable salts comprise acetate, methylester, HCl, sulfate, chloride and the like. The pharmaceutical compositions can be conveniently administered by any of the routes conventionally used for drug administration, for instance, orally, topically, parenterally or by inhalation. The substances may be administered in conventional dosage forms prepared by combining the drugs with standard pharmaceutical carriers according to conventional procedures. These procedures may involve mixing, granulating and compressing or dissolving the ingredients as appropriate to the desired preparation. It will be appreciated that the form and character of the pharmaceutically acceptable character or diluent is dictated by the amount of active ingredient with which it is to be combined, the route of administration and other well-known variables. The carrier(s) must be “acceptable” in the sense of being compatible with the other ingredients of the formulation and not deleterious to the recipient thereof. The pharmaceutical carrier employed may be, for example, either a solid or liquid. Exemplary of solid carriers are lactose, terra alba, sucrose, talc, gelatin, agar, pectin, acacia, magnesium stearate, stearic acid and the like. Exemplary of liquid carriers are phosphate buffered saline solution, syrup, oil such as peanut oil and olive oil, water, emulsions, various types of wetting agents, sterile solutions and the like. Similarly, the carrier or diluent may include time delay material well known to the art, such as glyceryl mono-stearate or glyceryl distearate alone or with a wax. The substance according to the present invention can be administered in various manners to achieve the desired effect. Said substance can be administered either alone or in the formulated as pharmaceutical preparations to the subject being treated either orally, topically, parenterally or by inhalation. Moreover, the substance can be administered in combination with other substances either in a common pharmaceutical composition or as separated pharmaceutical compositions. The diluent is selected so as not to affect the biological activity of the combination. Examples of such diluents are distilled water, physiological saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like. A therapeutically effective dose refers to that amount of the substance according to the invention which ameliorate the symptoms or condition. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

The dosage regimen will be determined by the attending physician and other clinical factors; preferably in accordance with any one of the above described methods. As is well known in the medical arts, dosages for any one patient depends upon many factors, including the patient's size, body surface area, age, the particular compound to be administered, sex, time and route of administration, general health, and other drugs being administered concurrently. Progress can be monitored by periodic assessment.

A typical dose can be, for example, in the range of 5 to 100 mg however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Progress can be monitored by periodic assessment. However, depending on the subject and the mode of administration, the quantity of substance administration may vary over a wide range to provide from about 1 mg per m² body surface to about 500 mg per m² body surface, usually 20 to 200 mg per m² body surface.

The pharmaceutical compositions and formulations referred to herein are administered at least once in accordance with the use of the present invention. However, the said pharmaceutical compositions and formulations may be administered more than one time, for example once weekly every other week up to a non-limited number of weeks.

Specific formulations of the substance according to the invention are prepared in a manner well known in the pharmaceutical art and usually comprise at least one active substance referred to herein above in admixture or otherwise associated with a pharmaceutically acceptable carrier or diluent thereof. For making those formulations the active substance(s) will usually be mixed with a carrier or diluted by a diluent, or enclosed or encapsulated in a capsule, sachet, cachet, paper or other suitable containers or vehicles. A carrier may be solid, semisolid, gel-based or liquid material which serves as a vehicle, excipient or medium for the active ingredients. Said suitable carriers comprise those mentioned above and others well known in the art, see, e.g., Remington's Pharmaceutical Sciences, Mack Publishing Company, Easton, Pa. The formulations can be adopted to the mode of administration comprising the forms of tablets, capsules, suppositories, solutions, suspensions or the like. The dosing recommendations will be indicated in product labeling by allowing the prescriber to anticipate dose adjustments depending on the considered patient group, with information that avoids prescribing the wrong drug to the wrong patients at the wrong dose.

In a further embodiment, the present invention relates to a kit for detection of any one of the aforementioned polynucleotides, the gene, the vector, the host cell, the transgenic non-human animal or the solid support. The kit may further comprise oligonucleotides or polynucleotides or probes capable of detecting the presence of the aforementioned polynucleotides, and optionally suitable means for detection and instructions for carrying out a method of the invention.

The kit of the invention may contain further ingredients such as selection markers and components for selective media suitable for the generation of transgenic cells and animals. The kit of the invention can be used for carrying out a method of the invention and could be, inter alia, employed in a variety of applications, e.g., in the diagnostic field or as research tool. The parts of the kit of the invention can be packaged individually in vials or other appropriate means depending on the respective ingredient or in combination in suitable containers or multicontainer units. Manufacture of the kit follows preferably standard procedures which are known to the person skilled in the art. The kit may be used for methods for detecting expression of genes or polynucleotides in accordance with any one of the above-described methods of the invention, employing, for example, nucleic acid hybridization and/or amplification techniques such as those described herein before and in the examples.

The explanations of the terms made herein above and in the following apply for all of the embodiments described below mutatis mutandis.

In a further embodiment the present invention relates to a method of diagnosing whether a subject has an extensive metabolizer (EM), intermediate metabolizer (IM) or poor metabolizer (PM) phenotype, comprising determining the absence or presence of one or more of the polynucleotides of the present invention.

The term “extensive metabolizer (EM), intermediate metabolizer (IM) or poor metabolizer (PM) phenotype” is an extensive metabolizer (EM), intermediate metabolizer (IM) or poor metabolizer (PM) phenotype of the CYP2D6 enzyme which is well known to the expert in the field and further described supra and in the literature including Bock, Pharmacogenetics 4 (1994), 209-218; Griese, Pharmacogenetics 8 (1998), 15-26.

The term “determining” encompasses means for direct or indirect determination of the absence or presence of the polynucleotides of the present invention. Direct determination encompasses techniques allowing specific detection of the polynucleotide of the invention itself. Such techniques encompass DNA sequencing, hybridisation techniques PCR based assays fluorescent dye and quenching agent-based PCR assay (Taqman PCR detection system), RFLP-based techniques, DNA sequencing-based techniques, hybridization techniques, single strand conformational polymorphism (SSCP), denaturating gradient gel electrophoresis (DGGE), temperature gradient gel electrophoresis (TGGE), chemical mismatch cleavage (CMC), heteroduplex analysis based system, techniques based on mass spectroscopy, invasive cleavage assay, polymorphism ratio sequencing (PRS), microarrays, a rolling circle extension assay, HPLC-based techniques, DHPLC-based techniques, oligonucleotide extension assays (OLA), extension based assays (ARMS, (Amplification Refractory Mutation System), ALEX (Amplification Refractory Mutation Linear Extension), SBCE (Single base chain extension), a molecular beacon assay, invader (Third wave technologies), a ligase chain reaction assay, 5′-nuclease assay-based techniques, hybridization capillary array electrophoresis (CAE), pyrosequencing, protein truncation assay (PTT), immunoassays and solid phase hydridization (dot blot, reverse dot blot, chips). Said techniques are very well known in the art and described, e.g., in Siitari, Nucleic acid diagnostics market, Technology Review 125/2002, ISDN 1239-758×, Caplin, Biochemica 1 (1999), 5-8; Neville, BioTechniques 32 (2002), 34-43; Shi 47 (2001), 164-72, Underhill, Genome Res 7 (1997), 996-1005; Oefner, J Chromatogr B Biomed Sci Appl 739 (2000), 345-55, the patent application US 20010049586. Moreover, kits for carrying out these techniques may be commercially available from, e.g., Applied Biosystems.

Further more, it might be necessary in connection with the determination referred to herein above to first amplify the polynucleotide of the invention, e.g., using a sample of genomic DNA from a subject to be analysed as a template. Moreover, further steps of nucleic acid treatments, such as reverse transcription of RNA samples, might be required and are also comprised by the term “determining” as used herein.

Specific methods for determining the polynucleotide of the invention are described below. Indirect detection can be carried out by detecting the presence or absence of the polypeptide encoded by the CYP2D6 gene of the present invention comprising the polynucleotide of the present invention. Said polypeptide is characterized in that it lacks an amino acid sequence encoded by exon 6 (SEQ ID No 22). As a result thereof, the polypeptide may be a truncated CYP2D6 polypeptide (SEQ ID No 23). The truncated polypeptide lacks CYP2D6 enzyme activity. Accordingly, the said polypeptide lacking the exon 6 encoded sequence might be detected directly, e.g., by antibody based methods, or indirectly by methods measuring an altered activity of the CYP2D6 enzyme activity. Such methods are well known in the art and described below.

The term “one or more polynucleotides” means that a subject may comprise in its genome a first polynucleotide of the present invention representing a first allele of the CYP2D6 gene and a second polynucleotide of the present invention representing a second allele of the CYP2D6 gene wherein said first and said second polynucleotide are different polynucleotides as specified herein above. Accordingly, such a subject would comprise more than one polynucleotides of the invention, namely two said polynucleotides of the invention. Of course, the further alleles of the CYP2D6 gene may also comprise polynucleotides of the present invention.

Based on the determination of the presence or absence of one or more of the polynucleotides of the present invention, the person skilled in the art is in a position to diagnose a CYP2D6 IM phenotype and therefore to distinguish an EM from an IM phenotype enabling an improved classification into the four phenotypic subgroups (PM, IM, EM and UM). A subject comprising one or more polynucleotides according to the present invention in its genome has an IM phenotype and, thus, not an EM phenotype. Accordingly, by determining the said presence or absence of one or more of the polynucleotides of the present invention in addition to the already known functional variants, e.g. PM and UM alleles, well known for the person skilled in the art (see, e.g., Gaedigk, Pharmacogenetics 9 (1999), 669-82; McElroy, AAPS PharmSci 2 (2000); Marez, Pharmacogenetics 7 (1997), 193-202; http://www.imm.ki.se/CYPalleles/cyp2d6.htm), it has become possible for the person skilled in the art to either directly diagnose the IM phenotype based on the detection of said polynucleotides or indirectly diagnose the EM phenotype based on the absence of the said polynucleotides. This now enables the person skilled in the art to select a defined number of individuals of each phenotypic subgroup (IM, PM, EM, UM) for the clinical development of new drugs to find the right dose of the drug for the patient, to avoid inefficacy or side effects of drug therapy or perform association studies to determine the clinical outcome of the therapy.

Furthermore, the present invention relates to a method of diagnosing whether a subject has an EM or IM phenotype comprising determining the absence or presence of one or more of the polynucleotides of the present invention.

In an other embodiment the present invention relates to a method of diagnosing whether a subject has an IM or PM phenotype, comprising determining the absence or presence of one or more of polynucleotides of the present invention.

Furthermore, the present invention also encompasses a method of determining whether an individual is at risk for a toxic reaction to treatment with a CYP2D6 substrate which comprises determining the absence or presence of one or more of the polynucleotides of the present invention.

The term “at risk for a toxic reaction to treatment with a CYP2D6 substrate” as used herein means that due to a reduced activity of the CYP2D6 enzyme the plasma concentration of a drug metabolized by CYP2D6 is higher in an individual compared to that of another individual without the presence of the variant polynucleotide. This might lead to a higher propensity to develop adverse reactions and/or toxic reactions at conventional doses, e.g. cardiovascular toxicity, induced peripheral neuropathy, oversedation, parkinsonism, tardive dyskinesis, movement disorders and other exptraypramidal side effects (Bertilsson, Br J Clin Pharmacol 53 (2002), 111-22; Eap, Pharmacogenetics 13 (2003), 39-47; Shimada, J Pharmacol Exp Ther 270 (1994), 414-23; Scordo, Pharmacogenomics 3 (2002), 201-18).

In a preferred embodiment of the present invention the substrate for which a toxic reaction is observed is selected from the group consisting of tricyclic antidepressants (e.g. amitriptyline, clomipramine, clopromazine, desipramine, imipramine, maprotiline, nortriptyline), selective serotonin reuptake inhibitors (SSRIs) (e.g. fluoxetine, fluvoxamine, mianserine, paroxetine), antipsychotics (e.g. chlorpromazine, zotepine), neuroleptics (e.g. haloperidol, perphenazine, risperidone, thioridazine, zuclopenthizoxol), anticancer agents (e.g. tamoxifen), beta adrenergic receptor antagonists (e.g. metoprolol, propanolol), antiarhythmic drugs (e.g. encainide, flecainide, mexiletine, propafenone, sparteine), amphetamines and opiates (e.g. codeine, dihydrocodeine, oxycodone, tramadol).

In another preferred embodiment the invention relates to a method of determining whether an individual is at risk for non-response to treatment with a CYP2D6 substrate which comprises determining the absence or presence of one or more of the polynucleotides of the present invention.

The term “is at risk for non-response to treatment with a CYP2D6 substrate” as used herein means a reduced formation of a pharmacologically active metabolite due to a reduced activity of the CYP2D6 enzyme in an individual compared to that of another individual without the presence of the variant polynucleotide leading to treatment failure with a CYP2D6 substrate.

In a still further embodiment, the present invention relates to a method of determining whether an individual is at risk for insufficient response to treatment with a CYP2D6 substrate which comprises determining the absence or presence of one or more of the of the present invention.

The term “at risk for insufficient response to treatment with a CYP2D6 substrate” as used herein means a reduced formation of a pharmacologically active metabolites due to a reduced activity of the CYP2D6 enzyme in an individual compared to that of another individual without the presence of the variant polynucleotide leading to inefficient treatment response with a CYP2D6 substrate.

Furthermore the present invention relates to a method of determining whether an individual is at risk for reduced metabolic activity of CYP2D6 to treatment with a CYP2D6 substrate which comprises determining the absence or presence of one or more of the polynucleotides of the present invention.

Another embodiment of the present invention encompasses a method of determining whether an individual is at risk for increased metabolic activity of CYP2D6 to treatment with a CYP2D6 substrate which comprises determining the absence or presence of one or more of the polynucleotides of the present invention.

In a preferred embodiment of the present invention, the above described method comprises the steps of:
(a) isolating a sample of biological material containing polynucleotides from a subject; and
(b) determining the absence or presence of one or more of the polynucleotides of the present invention.

As set forth above, the presence or absence of the polynucleotides of the present invention may also be determined by detecting the presence or absence of a polynucleotide or polypeptide lacking Exon 6 of the CYP2D6 gene. Specifically, if one or more polynucleotides are detected which lack Exon 6, this will be indicative for an IM or PM phenotype and/or the presence of a polynucleotide of the present invention. Thus, in a further embodiment the present invention comprises a method of diagnosing whether a subject has an EM, IM or PM phenotype comprising determining the absence or presence of Exon 6 (SEQ ID No: 22) of the CYP2D6 gene (SEQ ID No: 4).

In a preferred embodiment of the invention, the method of diagnosing whether a subject has an EM or IM phenotype comprises determining the absence or presence of Exon 6 (SEQ ID No: 22) of the CYP2D6 gene (SEQ ID No: 4).

In a more preferred embodiment the invention relates to a method of diagnosing whether a subject has an IM or PM phenotype comprising determining the absence or presence of Exon 6 (SEQ ID No: 22) of the CYP2D6 gene (SEQ ID No: 4).

The present invention also encompasses a method of determining whether a subject is at risk for a toxic reaction to treatment with a CYP2D6 substrate which comprises determining the absence or presence of Exon 6 (SEQ ID No: 22) of the CYP2D6 gene (SEQ ID No: 4).

The invention also relates to a method of determining whether a subject is at risk for non-response to treatment with a CYP2D6 substrate which comprises determining the absence or presence of Exon 6 (SEQ ID No: 22) of the CYP2D6 gene (SEQ ID No: 4).

In a further embodiment the present invention comprises a method of determining whether a subject is at risk for insufficient response to treatment with a CYP2D6 substrate which comprises determining the absence or presence of Exon 6 (SEQ ID No: 22) of the CYP2D6 gene (SEQ ID No: 4).

Moreover, the present invention relates to a method of determining whether a subject is at risk for reduced metabolic activity of CYP2D6 to treatment with a CYP2D6 substrate which comprises determining the absence or presence of Exon 6 (SEQ ID No: 22) of the CYP2D6 gene (SEQ ID No: 4).

In a preferred embodiment of the present invention, the above described method comprises the steps of:
(a) isolating a sample of biological material containing polynucleotides from a subject; and
(b) determining the absence or presence of Exon 6 (SEQ ID No: 22) of the CYP2D6 gene (SEQ ID No: 4).

In a more preferred embodiment of the invention, the above described method comprises the steps of:
(a) isolating a sample of biological material containing polypeptides from a subject; and
(b) determining the absence or presence of a polypeptide having an amino acid sequence as shown in SEQ ID No: 23.

In a further preferred embodiment of the present invention, the above described method is comprising PCR based techniques, fluorescent dye and quenching agent-based PCR assay (Taqman PCR detection system), RFLP-based techniques, DNA sequencing-based techniques, hybridization techniques, single strand conformational polymorphism (SSCP), denaturating gradient gel electrophoresis (DGGE), temperature gradient gel electrophoresis (TGGE), chemical mismatch cleavage (CMC), heteroduplex analysis based system, techniques based on mass spectroscopy, invasive cleavage assay, polymorphism ratio sequencing (PRS), microarrays, a rolling circle extension assay, HPLC-based techniques, DHPLC-based techniques, oligonucleotide extension assays (OLA), extension based assays (ARMS, (Amplification Refractory Mutation System), ALEX (Amplification Refractory Mutation Linear Extension), SBCE (Single base chain extension), a molecular beacon assay, invader (Third wave technologies), a ligase chain reaction assay, 5′-nuclease assay-based techniques, hybridization capillary array electrophoresis (CAE), pyrosequencing, protein truncation assay (PTT), immunoassays and solid phase hydridization (dot blot, reverse dot blot, chips). Said techniques are very well known in the art and described, e.g., in Siitari, Nucleic acid diagnostics market, Technology Review 125/2002, ISDN 1239-758X, Caplin, Biochemica 1 (1999), 5-8; Neville, BioTechniques 32 (2002), 34-43; Shi 47 (2001), 164-72, Underhill, Genome Res 7 (1997), 996-1005; Oefner, J Chromatogr B Biomed Sci Appl 739 (2000), 345-55, the patent application US 20010049586. Moreover, kits for carrying out these techniques may be commercially available from, e.g., Applied Biosystems.

Furthermore, the invention relates to the above described method wherein using oligonucleotides for the detection of a polynucleotide of the invention and/or genotyping of corresponding individual CYP2D6 variants of the invention. Preferably, said oligonucleotide is a polynucleotide of the invention described before. In a particular preferred embodiment said oligonucleotide is about 15 to 50, preferably 18 to 40, more preferably 18 to 28 nucleotides in length and most preferably said oligonucleotide comprises the nucleotide sequence of any one of SEQ ID NO: 5-21 or a complementary sequence.

The present invention furthermore relates to a method of selecting a subject suffering from a CYP2D6 substrate treatable disease for a treatment with said substrate said method comprising the steps of:
(a) determining the absence or presence of one or more of the polynucleotides of the present invention.
(b) determining based on the result obtained in step (a) whether the subject can be treated with the CYP2D6 substrate wherein the absence of the polynucleotide of the present invention is indicative for a treatment.

The term “CYP2D6 substrate treatable disease” means a disease in a subject that can or will be treated with a drug which is a substrate for the drug metabolizing enzyme CYP2D6. Many drug metabolizing enzymes are involved in the activation or inactivation of a medicament. In vivo and in vitro methods are used for the identification of CYP isoforms involved in the metabolism of the test item and to determine the contribution of each metabolizing enzyme in the metabolism of a drug. Those methods are well known in the art and encompass for example the use of primary or cryopreserved hepatocytes, individual liver microsomes, tissue slices (e.g. colon, intestine, liver, kidney), human recombinant enzymes (e.g. baculosomes®), the V79 Cell Battery™ which consists of a panel of recombinant V79 cell lines expressing a broad range of phase I and phase Ii enzymes relevant in the metabolism of xenobiotics and analytical methods such as LC-MC/MS detection, HPLC, fluorescent method detection and radiodetection.

Said CYP2D6 substrates are well known in the art such as alprenolol, aprindine, amiflamine, amitryptiline, ajmalin, brofaromine, bupranolol, bufuralol, carvedilol, chlorpromazine, cinnarizine, citalopam, clomipramine, codeine, debrisoquine, desipramine, dexfenfluramine, droperidol, ecstasy, encainide, flecainide, flunarizine, fluoxetine, flupheanzine, fluvoxamine, haloperidol, indoramine, imipramine, metoprolol, mexiletine, mianserine, mirtazepine, nefazodone, nortriptyline, ondansetron, paroxetine, perazine, perhexilene, perphenazine, phenacetin, phenformine, promethazine, pindolol, propafenone, propranolol, risperidone, sparteine, tamoxifen, thioridazine, timolol, tomoxetine, tramadol, tropisetron, venlafaxine, zotepine, and zuclopenthizol. Moreover, it is well known in the art for which diseases the above substrates are approved by the regulatory authorities. A list of such disease is disclosed in Mosby's DRUGConsult (2002), Mosby's Inc., St. Louis, Mo., USA and Rote Liste®, annual edition 2003, or from the package leaflets of the marketed products.

Inline of the foregoing, preferably, the diseases are congestive heart failure, hypertension, angina pectoris, depression, cardiac arrhythmias, paroxysmal supraventricular tachycardias, atrioventricular nodal reentrant tachycardia, atrioventricular reentrant tachycardia and other supraventricular tachycardias of unspecified mechanism, paroxysmal atrial fibrillation/flutter, prevention of ventricular arrhythmias, psychotic disorders, nausea and vomiting, restlessness and apprehension before surgery, acute intermittent porphyria, manic-depressive illness, intractable hiccups, combativeness and/or explosive hyperexcitable behavior in children, hyperactive children with excessive motor activity accompanying conduct disorders consisting of some or all of the following symptoms: impulsivity, difficulty sustaining attention, aggressivity, mood lability and poor frustration tolerance, obsessive-compulsive disorder, pain, obesity, to produce tranquilization, anxiety, bulimia nervosa, schizophrenia, childhood enuresis, panic disorder, social anxiety, breast cancer, supraventricular arrhythmias, paroxysmal atrial tachycardias, Wolff-Parkinson-White syndrome, sinus tachycardia tachycardias and arrhythmias due to thyrotoxicosis, atrial extrasystoles, atrial flutter and fibrillation, ventricular tachycardias. The term “treatment” as used herein encompasses treatment, amelioration or reduction of the degree of severity of the symptoms accompanied with the diseases, disorders or medical conditions to be treated. Treatment in the sense of the present inventions means that the symptoms of the diseases, disorders or medical conditions referred to herein will treated, ameliorated or reduced in a statistically significant number of subjects. Thus, treatment may also encompass single cases in which treatment is, in principle, not successful.

The explanations of the terms made herein above and in the following apply for all of the embodiments described below mutatis mutandis.

The determination of the CYP2D6 gene of humans with the methods referred to herein is important for the optimization of therapies and avoidance of adverse drug reactions or drug interactions with the numerous substrates of CYP2D6.

In a more preferred embodiment the present invention encompasses a method of selecting a subject suffering from a CYP2D6 substrate treatable disease for a treatment with said substrate comprising the steps of:
(a) determining the absence or presence of absence or presence of Exon 6 (SEQ ID No: 22) of the CYP2D6 gene (SEQ ID No: 4).
(b) determining based on the result obtained in step (a) whether the subject can be treated with the CYP2D6 substrate wherein the presence of Exon 6 (SEQ ID No: 22) of the CYP2D6 gene (SEQ ID No: 4) is indicative for a treatment.

In a further embodiment the invention relates to a method of treating a subject suffering from a CYP2D6 substrate treatable disease which comprises the steps of:
(a) determining the absence or presence of one or more of the polynucleotides of the present invention.
(b) administering to a subject lacking one or more of the polynucleotides of the present invention an effective amount of the CYP2D6 substrate.

In a more preferred embodiment the invention relates to a method of treating a subject suffering from a CYP2D6 substrate treatable disease which comprises the steps of:
(a) determining the absence or presence of Exon 6 (SEQ ID No: 22) of the CYP2D6 gene (SEQ ID No: 4).
(b) administering to a subject having Exon 6 (SEQ ID No: 22) of the CYP2D6 gene (SEQ ID No: 4) an effective amount of the CYP2D6 substrate.

Furthermore, the present invention relates to a method of treating a subject suffering from a CYP2D6 substrate treatable disease which comprises the steps of:
(a) determining the absence or presence of one or more of the polynucleotides of the present invention.
(b) determining an effective amount of the CYP2D6 substrate to be used for the treatment based on the result of step (a); and
(c) in a subject having one or more of the polynucleotides of the present invention as determined in step (a), administering the amount of the CYP2D6 substrate as determined in step (b).

Preferably, the invention relates to a method of treating a CYP2D6 substrate treatable disease which comprises the steps of:
(a) determining the absence or presence of Exon 6 (SEQ ID No: 22) of the CYP2D6 gene (SEQ ID No: 4).
(b) determining an effective amount of the CYP2D6 substrate to be used for the treatment based on the result of step (a); and (c) in a subject lacking Exon 6 (SEQ ID No: 22) of the CYP2D6 gene (SEQ ID No: 4) as determined in step (a), administering the amount of the CYP2D6 substrate as determined in step (b).

Moreover, the present invention relates to a method of identifying a diagnostic composition said method comprising the steps of:
(a) isolating a polynucleotide or the gene from a plurality of subgroups of individuals, wherein one subgroup has no prevalence for a CYP2D6 associated disease and at least one or more further subgroup(s) do have prevalence for a CYP2D6 associated disease; and
(b) identifying a single nucleotide polymorphism by comparing the nucleic acid sequence of said polynucleotide or said gene of said one subgroup having no prevalence for a CYP2D6 associated disease with said at least one or more further subgroup(s) having a prevalence for a CYP2D6 associated disease.

The term “prevalence” as used herein means that individuals are be susceptible for one or more disease(s) which are associated with a reduced expression and/or activity of the CYP2D6 enzyme or could already have one or more of said disease(s). Moreover, symptoms which are indicative for developing said disease are very well known in the art and have been sufficiently described in textbooks as cited above.

The term “identifying” as used herein encompasses the determination of the nucleic acid sequence of the polynucleotide isolated in step (a). Moreover, it encompasses comparing said polynucleotide with a reference sequence, preferably, the sequence shown in SEQ ID No: 4. Moreover, means for making a correlation between the occurrence of the single nucleotide polymorphism and the subgroup are included. Such means include for instance statistical tests, such as Student's t-test, chi²-test or the U-test according to Mann and Whitney, and technical means, such as computers and computer programs for carrying out said statistic test. Moreover, the person skilled in the art can adopt these and other statistical method known in the art individually without an undue burden.

Finally, the method should preferably comprise the step of formulating the polynucleotide identified in step (b) as a diagnostic composition, e.g. as a polynucleotide or oligonucleotide probe or other diagnostic compositions as specified herein above.

The invention relates to a method of diagnosing a disease related to the presence of a molecular variant of a CYP2D6 gene or susceptibility to such a disorder comprising determining the presence of a polynucleotide of the invention in a sample from a subject.

Moreover, the invention relates to a method of detection of the polynucleotide or the gene of the invention in a sample comprising the steps of:
(a) contacting the solid support described supra with the sample under conditions allowing interaction of the polynucleotide or the gene with the plurality of immobilized targets on the solid support and;
(b) determining the binding of said polynucleotide or said gene to said immobilized targets on the solid support.

The term “contacting” as referred to herein encompasses all techniques which enable a direct contact between the immobilized targets on the solid support and the polynucleotide or gene of the invention present in a sample. Preferably, contacting occurs in a liquid or gel or at least under humid atmosphere. The liquid or gel may be supplemented with a suitable buffer which allows or enhances interaction between the immobilized targets and the polynucleotides or genes of the invention present in the sample. Suitable liquids or gels for this purpose are well known in the art and are described in, e.g., Cheung, Nat. Genet. 21 (1999), 15-9. More preferably, electric fields are used to accelerate the contact between the immobilized target and the sample.

The term “conditions allowing interaction” refers, preferably, to those conditions under which a specific interaction takes place. Specificity of the interaction is, in principle, governed by ionic strength of the incubation liquid and temperature, electric fields or dependent on the agitation system used as disclosed for example in U.S. Pat. No. 6,287,850. The person skilled in the art can adjust suitable conditions for detection by routine experimentation. Preferably, the term “conditions allowing interaction” refers to reactions where polynucleotides can be bound by ligases or via chemical or photochemical reactions. For detection methods including fluorescence, chemiluminescence, mass spectrometry, and also conductivity and electronic methods, can be used as described for example in Watson, Current opinion in Biotechnology 9 (1998), 609-614.

The invention also relates to a method for diagnosing a disease comprising the steps of the method described supra, wherein binding of said polynucleotide or gene to said immobilized targets on said solid support is indicative for the presence or the absence of said disease or a prevalence for said disease.

In a preferred embodiment of the above described method said disease is codeine dependence, depression, hepatitis C, psychosis, schizophrenia, parkinsonism.

The invention will now be described by reference to the following biological Examples which are merely illustrative and are not constructed as a limitation of the scope of the present invention.

### EXAMPLE 1

Genomic samples, isolated by standard techniques from human blood samples were obtained from healthy Caucasian volunteers under consideration of all legal, ethical and medical requirement of the local ethical committee. Blood samples were obtained and processed by ion exchange chromatography methods (Qiagen) to isolate DNA.

All individuals included in this study had been previously phenotyped with sparteine according to published methods (Griese, Pharmacogenetics 8 (1998), 15-26; Sachse, Am J Hum Genet 60 (1997), 284-95; Bock, Pharmacogenetics 4 (1994), 209-18). Genotyping for the CYP2D6 polymorphisms including *2, *2×2, *3, *4; *5 or promoter variants of the CYP2D6 gene including −1584C>G is well known to the expert in the field and is further described in (Raimundo, Pharmacogenetics 10 (2000), 577-581) or indicated below. The base numbering was performed according to the Human Cytochrome P450 (CYP) Allele Nomenclature.

1. Description of Methods:

Differential Detection of CYP2D6*2 and CYP2D6*41 by Denaturing HPLC (DHPLC)

For analyzing the polymorphism at position 2988 of the CYP2D6 gene a nested polymerase chain reaction (PCR) was performed. Using primers localized at position 3187-3203 and 5016-4994 of the CYP2D6 gene (Genbank accession number M33388.1) first a 1830 bp product comprising the entire exons and introns 4, 5 and 6 was generated. Amplification were done in a 50 ul volume containing primers (0.4 μM), dNTP (200 μM), 100 ng of genomic DNA and the Expand High Fidelity Polymerase system (Boehringer Mannheim). Following 2 min heating at 95° C., thermal cycling of 30 sec at 95° C., 30 sec at 62° C. and 2 min at 72° C. was performed for 32 cycles and 5 min at 72° C. for 1 cycle on a PTC200 thermal cycler (MJ Research Inc., Watertown, Mass.). For analyzing the position 2988 of the CYP2D6 gene a 252 bp fragment comprising the entire exon 6 with flanking intronic sequences was specifically amplified from the 1830 bp product using the primers 2D6-F10 (5′-CTG TCC CGA GTA TGC TCT CG-3′, SEQ ID NO 5) and 2D6-4647-R (5′-GGT GTC CCA GCA AAG TTC ATG G-3′, SEQ ID NO 6). The amplification conditions were: 5 min 95° C., 1 cycle, 30 sec 92° C., 30 sec 60° C., 30 sec 72° C., 30 cycles, 7 min 72° C., 1 cycle.

The PCR products were denatured at 95° C. for 5 min, cooled down to 65° C. at −1° C./min, and then analyzed by denaturing HPLC (DHPLC). It is well known in the art how to perform the DHPLC method and is further referred to in Underhill, Genome Res 7 (1997), 996-1005; Oefner, J Chromatogr B Biomed Sci Appl 739 (2000), 345-55.

Analysis was performed using the WAVE™ DNA Fragment Analysis System (Transgenomic Inc., Omaha, Nebr., USA). The stationary phase consisted of a DNA Sep® Column System (Transgenomic Inc., Omaha, Nebr., USA) filled with alkylated nonporous poly(styrene divinylbenzene) particles. The column mobile phase consisted of a mixture of 0.1 M TEM, pH 7.0 without (bufferA) and with 25% acetonitril (buffer B). Unpurified PCR products were subjected to the preheated column and eluted with a linear acetonitrile gradient of 4.5 min from 53% to 62% buffer B at a flow rate of 0.9 ml per minute as proposed by the WAVEMAKER software. The melting temperature used was 63° C. as predicted by the DHPLC “melt” algorithm available at http://insertion.stanford.edu/melt.html.

Heteroduplex formation was detected from the melting profile in comparison to wild type and mutant controls which were confirmed by sequencing. All samples were reanalyzed by adding equal amounts of the wild type PCR product before denaturation to detect homozygous mutants.

Detection of 2988G>A by Sequence Analysis

Amplifications were done in a 50 μl volume containing the forward primer 5′-GAC TCT GTA CCT CCT ATC CAC GTC A-3′(SEQ ID No 7) and reverse primer 5′GGG TGT CCC AGC AAA GTT CAT-3′ (SEQ ID NO 8) (0.6 μM each), dNTP (200 μM), ˜60 ng of genomic DNA and a Taq Polymerase (Qiagen, Hilden). Following 2 min heating at 94° C., thermal cycling of 45 s at 94° C., 45 s at 60° C. and 2 min at 72° C. was performed for 34 cycles. PCR products were purified using a QIAquick PCR purification kit (Qiagen, Hilden) and directly sequenced by cycle sequencing using the ABI BigDye terminator cycle sequencing kit and the internal forward primer 5′-TCGGCCCTGCTCAGGC-3′ (SEQ ID NO 9). Sequencing reactions were determined using PE Biosystems' capillary 3700 DNA Analyzers (Foster City, Calif.). The sequences were analyzed for the presence of polymorphisms using the PHRED/PHRAP/POLYPHRED/CONSED software package (University of Washington, Seattle).

Alternative Primer Combinations for Analysis of 2988G>A

Genotyping of 2988G>A can also be performed using the primer combinations indicated in Table 1.

TABLE 1

Primer combinations for determining the absence or
presence of the variant 2988G>A using TaqMan- or
sequencing analysis.

| | SEQ ID | | SEQ ID |
|---|---|---|---|
| forward primer (5′-3′) | No | reverse primer (5′-3′) | No |
| CATAGTGGTGGCTGACCTGTTCTC | 010 | TGCCTCCTATGTTGGAGGAGGTC | 015 |
| CCCCGTTCTGTCCCGAGTATG | 011 | GCCCTGACACTCCTTCTTGCCT | 016 |
| GCTACCCCGTTCTGTCCCG | 012 | TGCCTCCTATGTTGGAGGAGGTC | 017 |
| GAAACAGTGCAGGGGCCG | 013 | GGTCAGGCTTACAGGATCCTGGT | 018 |
| CGTGAGCCCATCTGGGAAA | 014 | CCGGCCCTGACACTCCTTC | 019 |

CYP2D6 Genotype and Phenotype Analysis of a Family

For sequence analysis of intronic regions of the CYP2D6 gene, which had not been analyzed before, two DNA samples were selected from individuals with IM phenotype and genotypes *41/*5 and *41/*4, as well as two samples from EM individuals with genotypes *2/*5 and *2/*4. The IM and EM samples with *41/*4 were from the father and the mother of the family studied previously (Raimundo, Pharmacogenetics 10 (2000), 577-581). Sequence analysis of genomic DNA was carried out in both directions using a LICOR sequence analyzer and appropriately spaced primers to obtain overlapping sequences for all 8 introns.

2. Description of Results:

The genotypes of the five samples and novel polymorphic positions are presented in Table 2. In particular, the presence of SNPs 2850C>T (R296C) and 4180C>T (S486T) were confirmed in exons 6 and 9 of all four samples, respectively, whereas −1584G was only found in the two samples with *2 alleles, whereas those samples with a *41 allele had −1584CC genotype. Several novel intronic SNPs were detected. Surprisingly it was found that the novel SNP 2988G>A, in intron 6, was found to be a candidate for linkage to allele *41.

TABLE 2
Metabolic Ratio for sparteine (MRₛ), genotype of a
promoter and intron 6 polymorphism at position −1584
and 2988 of the CYP2D6 gene in a family

| | | | Position | Position | |
|---|---|---|---|---|---|
| DNA-Samples | MRₛ | Genotyp | −1584 | 2988 | Phenotype |
| Father | 1.72 | *41/*4 | CC | GA | IM |
| Mother | 0.82 | *2/*4 | GC | GG | EM |
| Daugther I | 3.31 | *41/*4 | CC | GA | IM |
| Daugther II | 2.57 | *41/*4 | CC | GA | IM |
| Daugther III | 0.73 | *2/*4 | GC | GG | EM |

To prove this association, a total of 304 DNA samples were analyzed from a genotyped sample collection, all of which were from individuals phenotyped with sparteine (phenotypes: 13 UM with MR<0.15; 236 EM with MR<1.2; 31 IM with 1,2<MR<20; and 24 PM with MR>20). Genotyping was performed by denaturing HPLC analysis of a 252 bp PCR fragment containing the entire exon 6 and parts of introns 5 and 6.

Four different DHPLC profiles were observed in the heterozygous condition and confirmed by sequencing selected samples to be specific for different genotypes observed at the exon 6/intron 6 boundary: a profile specific for homozygous wild-type; 2850T (*2); 2850T+2988A (*41), and 2850T+2939A (novel variant of *2). The novel SNP 2988G>A was found in 53/304 analyzed alleles, corresponding to a total allele of 8.75%. The entire study population was randomly selected.

To investigate the predictivity of this SNP for the IM phenotype, all individuals with genotype *2/*0 (N=57 including 4 individuals with *2×2/*0) and *41/*0 (N=17; *41 was defined as 2850T/−1584C) were compared (FIG. 1).

Comparing the linkage patterns in individuals with different CYP2D6 genotypes revealed that the mutation 2988 G>A is specifically associated with the CYP2D6*41 allele (FIG. 1). All 55 individuals with EM phenotype had the wild type G at position 2988 whereas 17/19 IMs had the variant A at position 2988. Most notably, two of the EM samples had genotypes *41/*0 and *41×2/*0 (marked by cross in FIG. 1). Thus, using 2988G/A as marker of *41, these two false positive predictions were avoided. Contrary, there were two exceptions among the IMs, characterized by 2988G (marked by G in FIG. 1). DHPLC analysis revealed a different profile in these two samples. Sequencing demonstrated that they had an additional SNP 2939G>A in inton 4 as well as a silent mutation 2291 G>A in exon 6.

3. Significance of the Finding of the Invention

About 10-15% of the population are phenotypically intermediate metabolizer (Griese, Pharmacogenetics 8 (1998), 15-26). These individuals have a dramatically reduced metabolic CYP2D6 activity which is almost as low as that of poor metabolizers (PMs). Several recent studies suggest that individuals with IM phenotype may be at comparable risk as PMs (Dalen, Pharmacogenetics 9 (1999), 697-706.; Platten, Clinical Pharmacology & Therapeutics 63 (1998), 552-560) in particular under continuous therapy (Rau, Pharmacogenetics 12 (2002), 465-72.). Importantly, the IM phenotype was shown to be not simply the consequence of a heterozygous condition for one null-allele and one functional allele. Only if the residual functional allele has a reduced enzyme activity compared to the wild-type allele, the individual has an impaired CYP2D6 IM phenotype (i.e. reduced metabolic activity). Patients with an intermediate metabolizer genotype receiving normal doses of CYP2D6 substrates are therefore at risk of developing severe toxic side effects or therapeutic failure comparable to those of poor metabolizers.

It is well known to the expert in the field that many drugs are insufficiently metabolized in PMs including tricyclic antidepressants, neuroleptics, several β-adrenoceptor blocking agents, antiarrhythmics, serotonin 5-HT₃-receptor antagonists and opioids. In addition several drugs are inhibitor of the CYP2D6 activity in vitro and in vivo. Concomitant administration of an inhibitor with other CYP2D6 substrates might further reduce the metabolic activity of CYP2D6 as was shown for drugs such as thioridazine (Llerena, Ther Drug Monit 23 (2001), 616-20.). The clinical consequences of inhibition could be drug interaction and/or adverse effects (i.e. cardiotoxicity) caused by higher than expected plasma concentration of the drug and/or metabolite. Therefore it is important to evaluate the clinical significance of an impaired metabolism of drugs metabolized by CYP2D6, and to avoid adverse effects by prior genotyping of the individuals before therapy.

So far, only the presence of the wild type sequence (−1584C) could be used as a marker for the identification of intermediate metabolizers. This marker has an allelic frequency of about 25% and is also common among extensive metabolizers. Therefore by itself this marker can not be used solely for the identification of intermediate metabolizers. Only the detection of a C or G in conjugation with at least two polymorphisms specific for the *2 allele (2850C>T and 4180C>T) allows to make a phenotype prediction. The novel SNP was almost exclusively detected on a genetic background of CYP2D6*2 with only one exception detected so far. Therefore the new mutation 2988G>A was found to be a better marker that is strongly associated with a higher metabolic ratio (lower enzymatic activity) of the CYP2D6 enzyme. With the identification of the new mutation of the invention an improved diagnosis now enables to better distinguish between extensive and intermediate metabolizers and enables to avoid false predictions.

### EXAMPLE 2

The expression of CYP2D6 mRNA from liver biopsies of individuals with a known genotype was analyzed by RT-PCR.

1. Description of the Methods

Preparation of cDNA

Blood and matching liver samples were collected during surgical interventions conducted at the Department of Surgery, University Medical Center Charité, Humboldt University in Berlin, Germany. The donors were Europeans of Caucasian origin. The liver samples included non-tumorous tissue surrounding primary liver tumors and metastases of various tumors or liver material surgically removed for other reasons.

For the preparation of high-quality RNA, a small tissue piece of 5 mm maximal side length was cut out immediately from each liver specimen and immediately transferred into a vial containing 1 ml of RNAlater™ (Ambion, Austin, Tex.).

Following homogenization, total RNA was isolated using RNeasy kit (Qiagen, Hilden, Germany). cDNA from liver samples as well as from the Clontech tissue panel was synthesized from 400 ng of total RNA using random hexamer primers (0.1 A₂₆₀ units), dNTPs (0.3 mM) and 50 U of Superscript Reverse Transcriptase (Life Technologies, Rockville, Md., USA) in a total volume of 30 μl using the buffer and reaction conditions provided by Life Technologies.

RT-PCR:

A 649 bp product was amplified using the 5′ primer from exon 5 (5′-ACT GAG GCC TTC CTG GCA GAG AT-3′, SEQ ID NO 20) and a 3′ primer of exon 9 (5′-ATG GGC TCA CCA GGA AAG CAA A-3′, SEQ ID NO 21). The amplification conditions were: 2 min 94° C., 1 cycle, 60 sec 92° C., 30 sec 69° C., 90 sec 72° C., 35 cycles, 7 min 72° C., 1 cycle.

Quantitation of CYP2D6 Splice Variant by DHPLC

The DHPLC method was used for quantification of the splice variant of CYP2D6 lacking exon 6. The relative intensity of the 507 bp fragment normalized to 18S rRNA (analyzed by TaqMan analysis according to Endrizzi, Anal Biochem 300 (2002), 121-31) was compared between groups of different CYP2D6 genotypes: *1/*1 (n=12), *1/*2 (n=9), *2/*2 (n=5), *2/*0 (n=9), *0/*0 (n=5), *41/*0 (n=2), *41/*41 (n=1), *41/*1 (n=3).

2. Description of the Results

To determine whether the intron 6 mutation (2988G>A) is responsible for alternative splicing, a CYP2D6 specific RT-PCR was established using primers within exon 5 and exon 9. In those samples with the mutation 2988G>A a 200 bp shorter RT-PCR product was detected (FIG. 2). Cloning and sequencing of this 507 bp fragment lead to the identification of a splice product lacking the entire exon 6 of the CYP2D6 gene.

This splice variant has already been described by Huang et al. (Huang, Arch Biochem Biophys 343 (1997), 101-8.). These authors have analyzed the expression of CYP2D6 mRNA in lung tumor tissue in comparison to normal lung tissue, but could not detect a difference in the expression level in both tissues. Woo et al. have identified this splice variant in the brain. However, nobody has analyzed the genetic basis for the splice variant (Woo, Neurology 53 (1999), 1570-2.)

To further confirm The mechanism by which the 2988G>A change may lead to lower expression of CYP2D6 was investigated in a bank of genotyped human livers. 46 human liver samples genotyped for CYP2D6 alleles have been analyzed for the presence of the splice variant compared to wild type CYP2D6. These data have shown that there is a significant correlation between the ratio of the 507 bp/649 bp fragment normalized to 18S RNA of *41/*0 and *41/*41 vs all other genotypes (p<0,005, t-test).

3. Significance of the Invention

By RT-PCR analysis it could be shown that RNA from livers with *41 (2988A) alleles in comparison to *2 and *1 alleles have decreased amounts of normally spliced product but increased amounts of a splice variant that completely lacks exon 6 as shown by sequence analysis. Lack of exon 6 leads to frame-shift and premature translation termination and may thus explain the association of *41 with the intermediate metabolizer phenotype. The 2988 G>A SNP directly identifies *41 and therefore greatly simplifies CYP2D6 genotyping.

1. A polynucleotide comprising a polynucleotide selected from the group consisting of:
(a) a polynucleotide having the nucleic acid sequence of SEQ ID NO: 1, 2, or 3;
(b) a polynucleotide capable of hybridizing to a CYP2D6 gene, wherein said polynucleotide is having a substitution of at least one nucleotide at a position corresponding to position 4784, 4735, or 4087 of the CYP2D6 gene (SEQ ID NO 4); and
(c) a polynucleotide capable of hybridizing to a CYP2D6 gene, wherein said polynucleotide is having an A at a position corresponding to position 4784, 4735 or 4087 of the CYP2D6 gene (SEQ ID NO 4).

2. The polynucleotide of claim 1, wherein said polynucleotide is associated with an intermediate metabolizer (IM) phenotype of CYP2D6, codeine dependence, depression, hepatitis C, psychosis, schizophrenia, or parkinsonism.

3. (Canceled)

4. The polynucleotide of claim 1 which is DNA or RNA.

5. A gene comprising the polynucleotide of claim 1.

6. The gene of claim 5, wherein a nucleotide substitution results in altered expression of the variant gene compared to the corresponding wild type gene.

7. A vector comprising the polynucleotide of claim 1.

8. The vector of claim 7, wherein the polynucleotide is operatively linked to expression control sequences allowing expression in prokaryotic or eukaryotic cells or isolated fractions thereof.

9. A host cell comprising the polynucleotide claim 1.

10. A transgenic non-human animal comprising at least one polynucleotide of claim 1.

11. The transgenic non-human animal of claim 10 which is a mouse, a rat or a zebrafish.

12. A solid support comprising one or a plurality of the polynucleotide of claim 1 in immobilized form.

13. The solid support of claim 12, wherein said solid support is a membrane, a glass-or polypropylene- or silicon-chip, oligonucleotide-conjugated beads or a bead array, which is assembled on an optical filter substrate.

14. A composition comprising the polynucleotide of claim 1.

15. The composition of claim 14, which is a diagnostic composition or a pharmaceutical composition.

16. A diagnostic kit for detection of a single nucleotide polymorphism comprising the polynucleotide of claim 1.

17. A method of diagnosing whether a subject has an extensive metabolizer (EM), intermediate metabolizer (IM) or poor metabolizer (PM) phenotype, comprising determining the absence or presence of one or more of the polynucleotides of claim 1 or the absence or presence of Exon 6 (SEQ ID No: 22) of the CYP2D6 gene (SEQ ID No: 4).

18. A method of diagnosing whether a subject has an EM or IM phenotype comprising determining the absence or presence of one or more of the polynucleotides of claim 1 or the absence or presence of Exon 6 (SEQ ID No: 22) of the CYP2D6 gene (SEQ ID No: 4).

19. A method of diagnosing whether a subject has an IM or PM phenotype, comprising determining the absence or presence of one or more of the polynucleotides of claim 1 or the absence or presence of Exon 6 (SEQ ID No: 22) of the CYP2D6 gene (SEQ ID No: 4).

20. A method of determining whether an individual is at risk for a toxic reaction, non-response, insufficient response, or reduced metabolic activity of CYP2D6 to treatment with a CYP2D6 substrate which comprises determining the absence or presence of one or more of the polynucleotides of claim 1 or the absence or presence of Exon 6 (SEQ ID No: 22) of the CYP2D6 gene (SEQ ID No: 4).

21-23. (Canceled)

24. A method of any one of claims 17 to 20, wherein said method comprises the steps of:
(a) isolating a sample of biological material containing polynucleotides from a subject; and
(b) determining the absence or presence of one or more of the polynucleotides of claim 1, the absence or presence of Exon 6 (SEQ ID No: 22) of the CYP2D6 gene (SEQ ID No: 4), or the absence or presence of a polypeptide having an amino acid sequence as shown in SEQ ID No: 23.

25-33. (Canceled)

34. The method of any one of claims 17 to 20 comprising PCR based techniques, fluorescent dye and quenching agent-based PCR assay (Taqman PCR detection system), RFLP-based techniques, DNA sequencing-based techniques, hybridization techniques, single strand conformational polymorphism (SSCP), denaturating gradient gel electrophoresis (DGGE), temperature gradient gel electrophoresis (TGGE), chemical mismatch cleavage (CMC), heteroduplex analysis based system, techniques based on mass spectroscopy, invasive cleavage assay, polymorphism ratio sequencing (PRS), microarrays, a rolling circle extension assay, HPLC-based techniques, DHPLC-based techniques, oligonucleotide extension assays (OLA), extension based assays (ARMS, (Amplification Refractory Mutation System), ALEX (Amplification Refractory Mutation Linear Extension), SBCE (Single base chain extension), a molecular beacon assay, invader (Third wave technologies), a ligase chain reaction assay, 5′-nuclease assay-based techniques, hybridization capillary array electrophoresis (CAE), pyrosequencing protein truncation assay (PIT), immunoassays and solid phase hydridization (dot blot, reverse dot blot, chips).

35. A method of selecting a subject suffering from a CYP2D6 substrate treatable disease for a treatment with said substrate said method comprising the steps of:
(a) determining the absence or presence of one or more of the polynucleotides of claim 1 or the absence or presence of Exon 6 (SEQ ID No: 22) of the CYP2D6 gene (SEQ ID No: 4); and
(b) determining based on the result obtained in step (a) whether the subject can be treated with the CYP2D6 substrate wherein the absence of the polynucleotide of claim 1 or the presence of Exon 6 (SEQ ID No: 22) of the CYP2D6 gene (SEQ ID No: 4) is indicative for a treatment.

36. (Canceled)

37. A method of treating a subject suffering from a CYP2D6 substrate treatable disease which comprises the steps of:
(a) determining the absence or presence of one or more of the polynucleotides of claim 1 or the absence or presence of Exon 6 (SEQ ID No: 22) of the CYP2D6 gene (SEQ ID No: 4); and
(b) administering to a subject lacking one or more of the polynucleotides of claim 1 or a subject having Exon 6 (SEQ ID No: 22) of the CYP2D6 gene (SEQ ID No: 4) an effective amount of the CYP2D6 substrate.

38. (Canceled)

39. A method of treating a subject suffering from a CYP2D6 substrate treatable disease which comprises the steps of:
(a) determining the absence or presence of one or more of the polynucleotides of claim 1 or the absence or presence of Exon 6 (SEQ ID No: 22) of the CYP2D6 gene (SEQ ID No: 4);
(b) determining an effective amount of the CYP2D6 substrate to be used for the treatment based on the result of step (a); and
(c) in a subject having one or more of the polynucleotides of claim 1 or a subject lacking Exon 6 (SEQ ID No: 22) of the CYP2D6 gene (SEQ ID No: 4) as determined in step (a), administering the amount of the CYP2D6 substrate as determined in step (b).

40. (Canceled)

41. A method of identifying a diagnostic composition said method comprising the steps of:
(a) isolating a polynucleotide of claim 1 from a plurality of subgroups of individuals, wherein one subgroup has no prevalence for a CYP2D6 associated disease and at least one or more further subgroup(s) do have prevalence for a CYP2D6 associated disease; and
(b) identifying a single nucleotide polymorphism by comparing the nucleic acid sequence of said polynucleotide or said gene of said one subgroup having no prevalence for a CYP2D6 associated disease with said at least one or more further subgroup(s) having a prevalence for a CYP2D6 associated disease.

42. A method of diagnosing a disease related to the presence of a molecular variant of a CYP2D6 gene or susceptibility to such a disorder comprising determining the presence of a polynucleotide of claim 1 in a sample from a subject.

43. A method of detection of the polynucleotide of claim 1 in a sample comprising the steps of:
(a) contacting a solid support comprising one or a plurality of the polynucleotide of claim 1 with the sample under conditions allowing interaction of the polynucleotide of claim 1 with the plurality of immobilized targets on the solid support; and
(b) determining the binding of said polynucleotide or said gene to said immobilized targets on the solid support.

44. A method for diagnosing a disease comprising the steps of the method of claim 43, wherein binding of said polynucleotide or gene to said immobilized targets on said solid support is indicative for the presence or the absence of said disease or a prevalence for said disease.

45. The method of any one of claims 41 to 44, wherein said disease is codeine dependence, depression, hepatitis C, psychosis, schizophrenia, parkinsonism.
